(19) United States
(12) Patent Application Publication
(10) Pub. No.: US 2014/0005269 A1
(43) Pub. Date: Jan. 2, 2014

(54) POLYMERIC MATRIX OF POLYMER-LIPID NANOPARTICLES AS A PHARMACEUTICAL DOSAGE FORM

(71) Applicants: Ndidi Ngwuluka, Johannesburg (ZA); Viness Pillay, Johannesburg (ZA); Yahya Essop Choonara, Johannesburg (ZA); Lisa Claire Du Toit, Johannesburg (ZA)

(72) Inventors: Ndidi Ngwuluka, Johannesburg (ZA); Viness Pillay, Johannesburg (ZA); Yahya Essop Choonara, Johannesburg (ZA); Lisa Claire Du Toit, Johannesburg (ZA)

(73) Assignee: University of the Witwatersrand, Johannesburg, Johannesburg (ZA)

(21) Appl. No.: 13/989,462

(22) PCT Filed: Nov. 28, 2011
(86) PCT No.: PCT/IB2011/055340

§ 371 Date: Sep. 11, 2013

(30) Foreign Application Priority Data
Nov. 26, 2010 (ZA) 2010/03741

(51) Int. Cl. A61K 47/32; A61K 31/198

(52) U.S. Cl. CPC A61K 47/32; A61K 31/198
USPC 514/567; 514/772.1

(57) ABSTRACT

A pharmaceutical dosage form for the release of at least one pharmaceutically active ingredient is claimed. The pharmaceutical dosage form includes a polymer matrix, polymer-lipid nanoparticles incorporated within the matrix and the pharmaceutically active ingredient(s). The polymer matrix is formed from at least two crosslinked cationic and anionic polymers, such as Eudragit® E100 and sodium carboxymethlycellulose. It can also include a neutral polymer, such as one derived from locust bean. The polymer-lipid nanoparticles are formed from at least one polymer, such as Eudragit® E100 and/or chitosan, and at least one phospholipid, such as lecithin. The polymer(s) and phospholipid are crosslinking with a chelating agent, such as sodium tripolyphosphate. The active ingredient or ingredients can be any pharmaceutically active compound(s), and in particular poorly absorbed compounds such as levodopa for the treatment of Parkinson's disease.

### FIELD OF THE INVENTION

This invention relates to a pharmaceutical dosage form, an din particular to a pharmaceutical dosage form for delivering a pharmaceutically active ingredient with poor absorption to a human or animal.

### BACKGROUND OF THE INVENTION

The successful management and treatment of Parkinson's disease (PD) has remained a challenge despite the discovery of the disease many years ago. Anticholinergic drugs were the first drugs to be used in the symptomatic treatment of PD. However, in 1960, it was discovered that dopamine is depleted from the striatum of PD patients. Patients were then placed on oral dopamine treatment, but this was eventually found to be less efficacious because of its inability to cross the Blood-Brain Barrier (BBB).

Trial studies ultimately led to the discovery of levodopa (L-dopa), a dopamine precursor, which was injected into PD patients for the first time in 1961. However, the bioavailability and consequently the therapeutic efficacy were found to be significantly reduced by extensive metabolism of L-dopa, principally through decarboxylation, o-methylation, transamination, and oxidation. The product formed by combining an aromatic L-amino acid decarboxylase inhibitor such as carbidopa and benserazide with L-dopa was shown to reduce the side-effects of L-dopa by either decreasing the metabolism or the dose. Despite all these drawbacks and the fact there are several therapeutic agents for the management of PD, L-dopa still remains the gold standard and most effective agent for the initial treatment.

In order to improve on the drawbacks as well as the bioavailability of L-dopa, several drug delivery systems have been developed. The first immediate release drug delivery systems for L-dopa was a tablet composed of L-dopa in combination with carbidopa (Sinemet®, Merck & Co., Inc. Whitehouse Station, N.J., USA). Carbidopa is a peripheral dopa decarboxylase (DDC) inhibitor. Benserazide is another decarboxylase inhibitor which is used in combination with L-dopa as Madopar® (Madopar®, F.Hoffmann-La Roche Ltd, Basel, Switzerland). These combinations, namely Sinemet® and Madopar®, can reduce the peripherial metabolism of L-dopa and side-effects such as nausea and vomiting but are ineffective in controlling dyskinesias and motor fluctuations associated with long-term use of L-dopa. A triple combination of L-dopa, carbidopa and entacapone into a single tablet known as Stalevo® (Orion Pharma, Espoo, Finland) was approved by the US Food and Drug Administration (FDA) in 2003. However, entacapone increases dopaminergic side-effects such as dyskinesias thereby necessitating the L-dopa dose to be reduced.

To compensate for the reduced duration of clinical response experienced by immediate release drug delivery systems, oral disintegrating tablets were introduced in 2004. L-dopa oral disintegrating tablets (ODTs) enable the patient to take smaller and more frequent doses, which make it possible to tailor dosages to individual patient needs. Parcopa® (Schwarz Pharma, Inc., Milwaukee, Wis., USA), a commercially available ODT was approved by the US FDA in 2004. However, frequency of dosing leads to patient non-compliance and the desired constant delivery may not be achieved.

Liquid L-dopa formulations were introduced to facilitate rapid onset of action though their effects were observed to last for a very short period. Patients were observed to benefit from liquid L-dopa formulation within 5 minutes for a duration of 1-2 hours (Stacy, 2000). L-dopa liquid formulations are therefore given to reduce the delay in the ‘on’ effect which has been observed to be augmented by controlled release (CR) formulations. However, it has also been observed that although L-dopa liquid formulations may be independent of the gastric emptying rate, pulsatile delivery is often obtained instead of the desired constant delivery and it suffers non-compliance due to frequency of administration.

Reducing the interval between L-dopa doses through the administration of controlled release formulations was one of the approaches that was utilized to solve a “wearing off” problem encountered with L-dopa. CR formulations are often associated with a problem of variable bioavailability and consequently variable efficacy. Peak plasma levels are reached in about 2-4 hours after administration and peak concentrations may be lower than obtained with immediate release (IR) formulations. This may necessitate the patients to take the IR formulation in the morning and the CR formulation or combination IR and CR during the day in order to produce a rapid onset of action (Gasser et al., 1998). Sinemet® CR (L-dopa/carbidopa; Merck & Co., Inc. Whitehouse Station, N.J., USA) and Madopar® HBS (L-dopa/benserazide; F.Hoffmann-La Roche Ltd, Basel, Switzerland) are the two major conventional CR formulations currently available in the market

To overcome the delayed action of controlled drug delivery systems, dual release (DR) formulations were introduced (Rubin, 2000). Madopar® DR (SkyePharma, London, UK) is a DR formulation containing L-dopa and benserazide currently available in the market and was developed in the ratio of 4:1 of L-dopa/benserazide. Madopar® DR combines the advantages of a rapid onset of efficacy as well as a sustained effect. When DR formulations were compared with CR formulations, the mean Dyskinesia Rating Scale severity score was similar for both formulations (2.8±2.5 vs. 2.7±3.1) which may imply that there may be variable bioavailability with DR formulations as well.

Gastroretentive drug delivery systems have also been developed which include multiple-unit sustained release floating minitabs which have shown to float in vitro after 12 minutes, remain afloat for >13 hours and exhibit sustained-release with no ‘burst effect’ over 8 hours. An improvement on the formulation provided sustained release for more than 20 hours. However, the efficacy of the floating minitabs may not be much different from the hydrodynamically balanced systems (HBS).

An L-dopa-loaded unfolding multilayer delivery system was developed which was administered to beagle dogs. The gastroscopy showed that it unfolded to its extended size 15 minutes after administration and maintained the extended size for at least 2 hours. Overall, the study showed that the unfolding CR gastroretentive drug delivery formulation can achieve prolonged absorption and sustained blood levels of L-dopa. However, there is the risk of unfolding systems residing longer than desired in the gastric region of humans, making them ineffective for chronic therapy.

Although L-dopa remains the most effective anti-parkinsonian agent that is eventually required by all PD patients, it does not provide an optimal clinical response due to inability of these delivery systems to provide constant and sustained delivery of L-dopa over a prolonged period which would lead to optimal absorption and subsequent central nervous system (CNS) bioavailability. Furthermore, although alternative routes of administration of L-dopa have been explored (such as pulmonary, rectal, intravenous, transdermal and intraduodenal), the oral route remains the most convenient route of administration for chronic drug therapy.

Therefore, the development of more simplified treatment modalities employing an oral formulation that improves the absorption and subsequent bioavailability, with constant therapeutic plasma concentrations, of L-dopa, L-dopa in combinations with carbidopa or L-dopa in combination with benserazide is needed.

### SUMMARY OF THE INVENTION

According to a first aspect of the invention, there is provided a pharmaceutical dosage form for the release of at least one pharmaceutically active ingredient, the pharmaceutical dosage form comprising:
a polymer matrix formed from at least two crosslinked polymers,
polymer-lipid nanoparticles incorporated within the matrix and formed from at least one polymer and at least one phospholipid, and
at least one pharmaceutically active ingredient.

The pharmaceutically active ingredient(s) may be included in the polymer-lipid nanoparticles and/or may be included in the polymer matrix. For example, one pharmaceutically active ingredient may be included in the polymer-lipid nanoparticles and another may be included in the polymer matrix. One of the pharmaceutically active ingredients may be intended for release in the small intestine of a human or animal and the other may be intended for release in the gastric region.

The two crosslinked polymers which make up the polymer matrix may be a cationic polymer and an anionic polymer. The cationic polymer may be acid-soluble and it may be poly(butyl methacrylate-co-(2-demethylaminoeethyl) methacrylate-co-methyl methacrylate) 1:2:1. The anionic polymer may be water-soluble and it may be sodium carboxymethylcellulose.

A neutral polymer may also be used to make up the polymer matrix. The neutral polymer may be a galactomannan polymer and it may be derived from locust bean.

The combination of the polymers may render the dosage form gastroretentive.

The polymer used to form the polymer-lipid nanoparticles may be poly(butyl methacrylate-co-(2-demethylaminoeethyl) methacrylate-co-methyl methacrylate). Alternatively, the polymer may be chitosan and further alternatively the polymer may be a combination of poly(butyl methacrylate-co-(2-demethylaminoeethyl) methacrylate-co-methyl methacrylate) and chitosan. The phospholipid in the polymer-lipid nanoparticles may be lecithin.

A chelating agent may also be used to form the polymer-lipid nanoparticles, and the chelating agent may be sodium tripolyphosphate.

The polymer matrix of the pharmaceutical dosage form may be capable of swelling in a controlled manner when ingested and this swelling may cause the release of the pharmaceutically active ingredient by diffusion out of the matrix. The diffusion of the pharmaceutically active ingredient may occur in a zero-order manner. The polymer matrix may also include an additive to further increase the ability of the matrix to swell. This additive may be a polysaccharide polymer and in particular this polysaccharide polymer may be pullulan.

The pharmaceutically active ingredient may be L-dopa, or it may be a combination of L-dopa and carbidopa, a combination of L-dopa and benserazide or a combination of L-dopa, carbidopa and benserazide.

The pharmaceutical dosage form may be for use in the treatment of Parkinson's disease

According to a second aspect of the invention, there is provided a method of preparing a pharmaceutical dosage form substantially as described above, the method comprising the steps of:
synthesizing a polymer matrix by crosslinking at least two polymers,
synthesizing polymer-lipid nanoparticles from at least one polymer and at least one phospholipid,
incorporating the polymer-lipid nanoparticles into the polymer matrix, and
incorporating at least one pharmaceutically active ingredient into the polymer matrix or the polymer-lipid nanoparticles.

According to a third aspect of the invention, there is provided the use of a pharmaceutical dosage form as described above in a method of manufacturing a medicament for use in a method of treating a disease or condition. The pharmaceutically active ingredient may be L-dopa, or it may be a combination of L-dopa and carbidopa, a combination of L-dopa and benserazide or a combination of L-dopa, carbidopa and benserazide. The disease may be Parkinson's disease.

According to a fourth aspect of the invention, there is provided a method of treating Parkinson's disease, the method comprising administering to a patient in need thereof a dosage form substantially as described above, wherein the dosage form contains a therapeutically effective amount of L-dopa, L-dopa and carbidopa, L-dopa and benserazide or L-dopa, carbidopa and benserazide.

### BRIEF DESCRIPTION OF THE DRAWINGS

FIG. 1: shows FTIR spectra of a) native chitosan (CHT), b) native Eudragit (EUD), c) EUD/CHT nanoparticles and d) EUD nanoparticles.

FIG. 2: shows scanning electron microscopic images of levodopa-loaded polymethacrylate copolymer/chitosan poly-lipo nanoparticles: (a) magnification ×5000; and (b) magnification ×5500.

FIG. 3: shows images of a) EUD/CHT crosslinked with lecithin, b) multi-crosslinked EUD nanoparticles (×32), and TEM images of c) polymer-lipid nanoparticles (×8000) and d) polymer-lipid nanoparticles (×20000).

FIG. 4: shows surface morphology of the directly compressed IPB matrices a) mag×173; and b) Mag×10,178 showing the granules of the matrix components and crystals of levodopa; c) surface morphology of hydrated and lyophilized IPB matrices showing the pores left after sublimation of water molecules during lyophilization. Mag×168.

FIG. 5: shows a linear Isothermic plot—Nitrogen adsorption (+—red) and desorption (o—wine red) isotherms of interpolymeric blend.

FIG. 6A: shows FTIR spectra for interpolymeric blends (IPBs) formed according to the invention by cross-linking at least two polymers: a) native LB, EUD and CMC, b) Formulations E1-E10, c) Formulations E1-E3.

FIG. 6B: shows FTIR spectra for IPBs: d) Formulation E1 in varying normality's of acetic acid and e) Formulation E3 in varying normality's of acetic acid.

FIG. 7: shows typical Force-Distance and Force-Time profiles of the IPBs for determining a) matrix hardness and deformation energy and b) matrix resilience.

FIG. 8: shows (a) Interpolymeric tablet matrix loses (b) its three-dimensional shape as the pH increases to 4.5 after dissolution studies.

FIG. 9: shows (a) interpolymeric tablet matrix shape retained (b) its three-dimensional shape in pH 4.5 when polymeric nanoparticles are incorporated into it.

FIG. 10: shows magnetic resonance images of the mechanical behavioral changes of matrices in different pHs: (A) nanoparticles incorporated into interpolymeric blend at pH 1.5; (B) interpolymeric blend matrix without nanoparticles at pH 4.5 (C) nanoparticles incorporated into interpolymeric blend at pH 4.5 at 0, 3, 6, 9 and 12 h.

FIG. 11: shows a typical gastro-adhesive Force-Distance profile of the IPB matrices.

FIG. 12: shows gastro-adhesive profiling of Formulation E3 in varying normality's of acetic acid employing an applied force of 1N.

FIG. 13: shows gastro-adhesive profiling of Formulations E1-E10 employing an applied force of 1N.

FIG. 14: shows gastro-adhesive profiling for Formulation E3 in varying normality's of acetic acid employing an applied force of 0.5N.

FIG. 15: shows epithelial adhesive profiling of Formulation E1 in varying normality's of acetic acid employing an applied force of 0.5N.

FIG. 16: shows epithelial adhesive profiling of Formulation E1 in varying normality's of acetic acid employing an applied force of 0.5N.

FIG. 17: shows profiles of the degree of swelling for Formulation E₃ in varying normality's of acetic acid.

FIG. 18: shows drug release profiles for Formulations E1-E10 employing 0.1N HCl as the dissolution medium.

FIG. 19: shows drug release profiles for Formulation E1 in different normality's of acetic acid employing 0.1N HCl as the dissolution medium.

FIG. 20: shows drug release profiles for Formulation E3 in varying normality's of acetic acid employing 0.1N HCl as the dissolution medium.

FIG. 21: shows drug release profiles for Formulation E3 in varying normality's of acetic acid employing buffer pH 1.5 (standard buffer KCl/HCl) as the dissolution medium.

FIG. 22: shows drug release profiles for Formulation E3 in varying normality's of acetic acid employing buffer pH 4.5 (0.025M KH₂PO₄/H₂PO₄) as the dissolution medium.

FIG. 23: shows comparative drug release profiles of levodopa from IPB matrices, Madopar® HBS capsules and Sinemet® CR.

FIG. 24: shows drug release profiles of polymer-lipid nanoparticles embedded within the IPB matrices employing buffer pH 1.5 (standard buffer KCl/HCl) as the dissolution medium.

FIG. 25: shows drug release profiles of polymer-lipid nanoparticles embedded within the IPB matrices employing buffer pH 4.5 (0.025M KH₂PO₄/H₂PO₄) as the dissolution medium.

### DETAILED DESCRIPTION OF THE INVENTION

The invention provides a pharmaceutical dosage form or composition for the release of at least one pharmaceutically active compound or ingredient. The pharmaceutical dosage form includes a polymer matrix, polymer-lipid nanoparticles incorporated within the matrix and the pharmaceutically active ingredient(s).

The polymer matrix is typically an interpolyelectrolyte complex formed from at least two crosslinked polymers. One of the polymers can be a cationic polymer, and is typically an acid-soluble polymer such as one based on dimethylaminoethyl methacrylate, butyl methacrylate and methyl methacrylate (e.g. poly(butyl methacrylate-co-(2-demethylaminoeethyl) methacrylate-co-methyl methacrylate) 1:2:1, commercially available as Eudragit® E100. The other polymer can be an anionic polymer that is preferably water soluble, such as sodium carboxymethlycellulose. A neutral polymer, typically a gallactomannan polymer such as one derived from locust bean can also be incorporated into the polymer matrix.

The cationic and anionic polymers are typically blended in a ratio of about 0.5:1, yielding a gel-like structure, or hydrogel, that is slowly degradable.

The polymer-lipid nanoparticles are formed from at least one polymer and at least one phospholipid. Suitable polymers are cationic acrylate-type polymers such as poly(butyl methacrylate-co-(2-demethylaminoeethyl) methacrylate-co-methyl methacrylate 1:2:1 (Eudragit E100) or cationic polysaccharide-type polymers such as chitosan, or a combination thereof. A suitable phospholipid is lecithin. The nanoparticles are formed by combining the polymer(s) and phospholipid and crosslinking them with a chelating agent, such as sodium tripolyphosphate. Other crosslinking agents such as a salt or sequestrator can also be used. The polymer-lipid nanoparticles which are formed are generally spherical with inner and outer cores. The nanoparticles can be hollow spherical nanocapsules.

One or more pharmaceutically active ingredient can be incorporated into the polymer and phospholipid solution to generate nanoparticles which are loaded with the active ingredients.

The nanoparticles and/or pharmaceutically active ingredients can be mixed with the polymer matrix or can be added to the mixture of the at least two polymers before the matrix forms. Similarly, one or more pharmaceutically active compounds, compositions or ingredients can also be mixed with the polymer matrix or can be added to the mixture of the two or more polymers before the matrix forms. In particular, where the dosage form contains two or more pharmaceutically active ingredients for release at different rates or in different sites, the nanoparticles can be loaded with one active ingredient and the polymer matrix can be loaded with another active ingredient. For example, the active pharmaceutical ingredient incorporated within the polymer-lipid nanoparticles can be a compound which is intended to be released within the small intestine of a subject, while the other active pharmaceutical ingredient that is incorporated within the polymer matrix can be a compound which is intended to be released within the gastric region of a subject.

The active ingredient or ingredients can be any pharmaceutically active compound(s), and is typically a compound which is poorly absorbed by the human or animal body, such as a narrow window absorption drug.

The pharmaceutical dosage form can be formed so as to be administrable via any one of oral, subcutaneous, vaginal, rectal or transdermal routes for the rate-modulated, site-specific delivery of various active pharmaceutical ingredients.

In a particular embodiment, the dosage form can be prepared by mixing and blending the polymer matrix, the nanoparticles and optionally additional active ingredients such as excipients and additives, and compressing the mixture to produce high density, swelling and bioadhesive polymer-lipid nanoparticle-loaded controlled release gastroretentive drug delivery systems (CR-GRDDS).

In the same or a different embodiment of the invention, the dosage form can be a drug delivery system which controls and targets the release of anti-Parkinson's disease drugs for the treatment of Parkinson's disease. The drugs can be levodopa (L-dopa), L-dopa and carbidopa, L-dopa and benserazide or L-dopa, carbidopa and benserazide.

In one embodiment, the dosage form contains L-dopa as the active ingredient and is for the treatment of PD. In another embodiment, the dosage form contains L-dopa in combination with carbidopa. In yet another embodiment the dosage form contains L-dopa in combination with benserazide. CR-GRDDS are preferred for the present invention to the traditional dosage forms for drugs that have confined sites of absorption, such as L-dopa. The site specificity for absorption is due to the low solubility of the drugs at the pH found in the lower gastro intestinal tract (GIT), enzymatic breakdown, drug degradation by micro flora in the colon, chemical instability of the drug and binding of the drug to the contents of the GIT. CR-GRDDS of the present invention are able to retain such drugs in the stomach over a prolonged period above the absorption window of these drugs to ensure suitable absorption and bioavailability, target drugs required at the stomach or proximal small intestine, reduce erratic concentrations of drugs or adverse effects and enhance therapeutic efficacy. The dosing frequency can therefore be reduced, and patient compliance with the treatment regime is therefore more likely to occur.

The polymer matrix can have modifiable physicochemical and physicomechanical properties which can provide for the rate-modulated diffusion, mechano-transduction and release of the nanoparticles to release the pharmaceutically active ingredients entrapped therein. The polymer matrix is able to control the release of the active pharmaceutical ingredients at rate-modulated kinetics, preferably at zero-order release kinetics over a prolonged period by mechanisms such as swelling modulation. The polymer matrix is also capable of retaining its three dimensional network and shape with robust mechanical strength.

The polymer matrix can swell in a controlled manner when ingested and this swelling causes the release of the nanoparticles by diffusion out of the matrix, and subsequent release of the pharmaceutically active ingredient(s). The matrix can swell to greater than 4 times its original size, for example >100% by weight after 1 hour, >350% after 12 hours and >450% after 24 hours.

The polymeric nanoparticles in the matrix enhances the mechanical strength of the matrix at higher pH values such as 4.5 and 6.8, which otherwise would have lost its three-dimensional network.

The elucidation of the physicochemical and physicomechanical properties of the dosage form of the present invention is described in the examples which follow. To improve the absorption and bioavailability of L-dopa over a prolonged period at a constant rate of delivery, the applicant has developed novel CR-GRDDS into which novel polymer-lipid nanoparticles are incorporated with a triple-mechanism approach. Miscible polymers in interaction with a phospholipid as a lipid component are multi-crosslinked with a first crosslinking agent and optionally a sequestrator as a second crosslinking agent to fabricate polymer-lipid nanoparticles. The polymer-lipid nanoparticles are embedded in an interpolymeric blend (IPB) generated by synthesizing an inter-polyelectrolyte complex comprising two polymers into which a third polymer is optionally incorporated. The IPB is produced by a simple, efficient and reproducible technique involving homogenous blending facilitated by salt generation with subsequent lyophilization and milling. The polymer-lipid nanoparticles are incorporated into the IPB and directly compressed with other additives or excipients to produce high density, swelling and bioadhesive poly-lipo nanoparticles loaded CR-GRDDS.

Dosage forms of the present invention have a triple-mechanism of action:
They are gastro-retentive due to swelling;
They have a zero order release;
They have preferential absorption because of the lip nanoparticles.

The matrix also protects the nanoparticles.

The physicochemical and physicomechanical properties of the dosage forms prepared according of the present invention were studied.

In the examples which follow, L-dopa was used as an example of a suitable active ingredient in order to design a CR-GRDDS which provides absorption and bioavailability of an active ingredient over a prolonged period at a constant rate of delivery. However, it will be apparent to a person skilled in the art that other active compounds could be used in the dosage form of the present invention and that L-dopa, L-dopa/carbidopa, L-dopa/benserazide and L-dopa/carbidopa/benserazide are just examples hereof. Other polymers and phospholipids could also be used to form the polymer-matrix and polymer-lipid nanoparticles, and are not only limited to those provided herein.

### Examples

### Materials and Methods

### Materials

Eudragit E100® (EUD) (Evonik Röhm GmbH & Co. KG, Darmstadt, Germany), sodium carboxymethylcellulose (CMC) (Sigma-Aldrich Chemie GmbH, Buchs, Switzerland), 3-(3,4-dihydroxyphenyl)-L-alanine (Sigma-Aldrich Inc, Steinheim, Germany), acetic acid glacial (Rochelle Chemicals, South Africa), hydrochloric acid (HCl) (Rochelle Chemicals, South Africa), locust bean (LB) from Ceratonia siliqua seeds (Sigma-Aldrich Inc, Steinheim, Germany), barium sulphate (BaSO₄), potassium phosphate monobasic (KH₂PO₄), pullulan from Aureobasidium pullulans (Sigma-Aldrich Inc, Steinheim, Germany), sodium hydroxide (NaOH), chloroform (Rochelle Chemicals, South Africa), silica, potassium chloride (KCl) (Saarchem, South Africa), magnesium stearate (Merck Chemicals (Pty) Ltd., South Africa), ortho-phosphoric acid (BDH Chemicals, Poole, England), chitosan (CHT) (food grade, Wellable group, Fujian, China), sodium tripolyphosphate (TPP) (Sigma-Aldrich Inc, Steinheim, Germany) and lecithin (Lipoid EPCS, Lipoid AG, Ludwigshafen, Germany).

### Synthesis of Polymer-Lipid Nanoparticles

Weighed quantities of EUD and varying quantities of EUD with CHT were dissolved in 10 mL 0.2N HCl and 100 mg of L-dopa was added into the polymeric solution. Lipoid EPCS (100 mg) was dissolved in 1 mL of chloroform and added to the L-dopa-loaded polymeric solution under mechanical agitation for 10 minutes. Varying concentrations of TPP dissolved in 0.2N acetic acid were added under agitation for another 10 minutes and thereafter lyophilized for 48 hours.

### Analysis of Particle Size and Surface Charge of the Polymer-Lipid Nanoparticles

Nanoparticle size, size distribution profiles and zeta potential were generated using a ZetaSizer NanoZS (Malvern Instruments, Malvern, UK) instrument equipped with non-invasive backscatter technology set at an angle of 173°. The nanoparticles sizes and zeta potentials were profiled after addition of lecithin, then after addition of TPP and finally after lyophilization.

### Analysis of Chemical Structure Variation of the Polymer-Lipid Nanoparticles

FTIR spectra over the range of 4000-650 cm⁻¹ were obtained for the native polymers employed and the polymer-lipid nanoparticles using a PerkinElmer spectrometer (PerkinElmer Spectrum 100, Beaconsfield, United Kingdom) to elucidate the chemical structural transitions which occurred during nanofabrication.

### Computational Modelling, Determination of pH and Absorbance Changes During Fabrication of Poly-Lipo Nanoparticles

To explicate the interactions between the polymers and crosslinking agents as well as the mechanisms of nanoparticle formation, computational modelling was undertaken. Models and graphics depicting the mechanisms of interactions were obtained using ACD/I-Lab, V5.11 (Add-on) software (Advanced Chemistry Development Inc., Toronto, Canada, 2000) while the possible interactions were assessed by using some general chemistry concepts and chemometric modeling concepts. Molecular mechanics computation in vacuum was undertaken using HyperChem™ 8.0.8 Molecular Modeling System (Hypercube Inc., Gainesville, Fla., USA) and ChemBio3D Ultra 11.0 (CambridgeSoft Corporation, Cambridge, UK). Changes in pH and absorbances were determined at each stage of incorporation of substances as described in the methodology for fabrication of poly-lipo nanoparticles. The pHs and absorbances were determined when the polymers were added to 0.2N HCl and afterwards when lecithin and TPP were added. The absorbances were obtained in the absence of L-dopa.

### Assessment of the Surface Morphology of the Polymer-Lipid Nanoparticles

The surface morphological analyses of the polymer-lipid nanoparticles were undertaken by performing digital microscopy. The digital microscopic images of the polymer-lipid nanoparticles after synthesis were obtained using Olympus digital microscope; Olympus SZX-ILLD2-200 (Olympus Corporation, Tokyo, Japan). The particle shape was further viewed with transmission electron microscopy (TEM) (Jeol 1200 Ex, 120 keV TEM, Tokyo, Japan) for higher definition and resolution.

### Determination of Drug-Loading and Drug Entrapment Efficiency of the Polymer-Lipid Nanoparticles

Percentage drug-loading efficiency was determined gravimetrically to assess the capacity of the nanoparticles with regards to the quantity of drug loaded in the nanoparticles. The percentage drug-loading was calculated based on the weights of the incorporated drug and the nanoparticles employing Equation 1.

$$Drug Loading ( \% ) = \frac{Quantity of drug in nanoparticles}{Quantity of nanoparticles} \times 100 \qquad Equation 1$$

The drug entrapment efficiency was determined by dispersing the polymer-lipid nanoparticles in 0.1N HCl and the amount of the drug in the medium was assessed spectrophotometrically to obtain the quantity of drug in the polymer-lipid nanoparticles with respect to the quantity of drug used in the formulation employing Equation 2.

$$Drug entrapment efficiency ( \% ) = \frac{Actual Amount of drug}{Theoretical amount of drug} \times 100 \qquad Equation 2$$

### Microscopical Analysis of the Levodopa-Loaded Poly-Lipo Nanoparticles

Lyophilized poly-lipo nanoparticles were spread thinly on a carbon tape and coated with gold-palladium. The nanoparticles were viewed under SEM (JEOL-JEM 840 scanning electron microscope, Tokyo, Japan) at a voltage of 15 KeV and current of 6×10⁻¹⁰ Amp.

### Synthesis of the Interpolymeric Blend (IPB) for the Polymer Matrix of the Gastroretentive Drug Delivery System

EUD was milled and dissolved in 50 mL 0.1N acetic acid while CMC was dissolved in 50 mL distilled water. The transparent EUD solution was added into a transparent CMC solution and allowed to stir under vigorous agitation for 3 hours at ambient temperature. After 3 hours, LB was added and allowed to stir for 15-20 minutes. The interpolymeric blend (IPB) formed was lyophilized for 48 hours, milled and employed for direct compression. The ratios of the polymers within the IPB are shown in Table 1. IPBs E1 and E3 comprising EUD-CMC in the ratios of 1:0.5 and 0.5:1 respectively were further synthesized in 0.2, 0.4, 0.6, 0.8 and 1.0N acetic acid.

TABLE 1
Compositions of the polymers utilized in ten polymeric blends

| Formulation (ratios) | Eudragit (g) | Locust bean (g) | CMC (g) |
|---|---|---|---|
| E1 (1:1:0.5) | 1.68 | 1.68 | 0.84 |
| E2 (1:0.5:1) | 1.68 | 0.84 | 1.68 |
| E3 (0.5:1:1) | 0.84 | 1.68 | 1.68 |
| E4 (1:1:1) | 1.4 | 1.4 | 1.4 |
| E5 (2:1:0.5) | 2.4 | 1.2 | 0.6 |
| E6 (1:2:0.5) | 1.2 | 2.4 | 0.6 |
| E7 (0.5:1:2) | 0.6 | 1.2 | 2.4 |
| E8 (0.5:2:1) | 0.6 | 2.4 | 1.2 |
| E9 (2:0.5:1) | 2.4 | 0.6 | 1.2 |
| E10 (1:0.5:2) | 1.2 | 0.6 | 2.4 |

### Analysis of Chemical Structure Variation of the Interpolymeric Blend (IPB)

FTIR spectra were obtained for the native polymers and the IPB using a PerkinElmer spectrometer (PerkinElmer Spectrum 100, Beaconsfield, United Kingdom) over a range of 4000-650 cm⁻¹ to elucidate the structural modification of the IPB from the native polymers.

Direct Compression of the Interpolymeric Blend into Matrices

The IPB was directly compressed with additives and excipients as listed in Table 2 using a Carver Press (Carver Industries, USA) at 3 tons. Mixing of the components was undertaken in the following sequence: 1) quantities of IPB were added and blended in an alternate fashion with excipients; 2) silicon dioxide was blended first with some quantity of IPB followed by L-dopa, then pullulan and BaSO₄ while magnesium stearate was added last and blended continuously for 2 minutes thereafter.

TABLE 2
Composition of the directly compressed IPB matrices

| Components | Quantity + Overage (mg) per matrix |
|---|---|
| L-dopa | 100 |
| IPB (50%) | 500 |
| Pullulan (10%) | 100 |
| Magnesium stearate (0.5%) | 5.5 |
| Silica (silicon dioxide) (5%) | 50.5 |
| BaSO₄ | 234 |

### Determination of the Densities of the Matrices

The volume of each matrix was determined by obtaining the diameter and the thickness using a 0-150 mm electronic digital caliper while the weights were ascertained gravimetrically. Hence the density for each matrix was calculated having obtained the weights and volumes.

### Evaluation of the Physicomechanical Strength of the Matrices

The physicomechanical strength of the matrices was determined by Force-Distance profiles using a Texture Analyzer (TA) (TA.XT plus, Stable Microsystems, UK). The matrix hardness and deformation energy were determined with a 2 mm flat-tipped steel probe while matrix resilience was determined using a 36 mm cylindrical probe fitted to the TA. The data was captured through Texture Exponent Software (V3.2). The parameter settings that were employed are shown in Table 3.

TABLE 3
Parameter settings for the textural analysis of the matrices

| Parameters | Settings |
|---|---|
| Pre test speed | 1 mm/sec |
| Test speed | 0.5 mm/sec |
| Post test speed | 1 mm/sec |
| Compression force¹ | 40 N |
| Trigger type | Auto |
| Trigger force | 0.5 N |
| Compression strain² | 25% |

¹Employed for matrix hardness and deformation energy;
²Employed for matrix resilience

### Assessment of Mechanical Behaviour of Matrices by Magnetic Resonance Imaging

A magnetic resonance system (MARAN-IP) with digital MARAN DRX console (Oxford Instruments, Oxfordshire, UK) equipped with a compact 0.5 Tesla permanent magnet which was stabilized at 37° C. and a dissolution flow through cell was used for viewing of the mechanical behaviour of the matrices. The glass beads were used to fill the cone-like lower part of the cell to provide laminar flow at 16 mL/min of the solvents employed. The matrices were placed within the cell which in turn was positioned in a magnetic bore of the system. Acquiring of magnetic resonance images was undertaken hourly over 12 hours with Maran-i software under continuous solvent flow conditions with buffers pH 1.5 and 4.5 at different occasions. The image acquisition parameters are depicted in Table 4.

TABLE 4
Image acquisition parameters applied during magnetic resonance
imaging using MARAN-i

| S. No. | Parameter | Value |
|---|---|---|
| 1. | Imaging protocol | FSHEF |
| 2. | Requested gain (%) | 1.90 |
| 3. | Signal strength | 71.62 |
| 4. | Average | 2 |
| 5. | Matrix size | 128 |
| 6. | Repetition time (ms) | 1000.00 |
| 7. | Spin Echo Tau (ms) | 6.00 |
| 8. | Image acquired after | 60 min |
| 9. | Total scans | 64 |

### Surface Morphological Analysis of IPB Matrices

To assess the surface morphology of IPB matrices, matrix samples were mounted on aluminium stubs with the aid of carbon paste. Afterwards, the matrix was sputter-coated with gold-pallidium and then viewed under Quanta™ Scanning Electron Microscope (FEI Quanta 400 FEG (ESEM) FEI Company, Eindhoven, The Netherlands). The non-hydrated and hydrated IPB matrices were observed under the microscope. The hydrated IPB matrix was left in the buffer pH 1.5 for 24 hours, frozen at −70° C. for another day and lyophilized before viewing under the Quanta™ Scanning Electron Microscope.

### Porositometric Analyses of IPB Matrices

The surface area and porosity analyses of IPB matrices were performed using a porositometric analyzer (ASAP 2020, Micromeritics, Norcross, Ga., USA). The sizes that could fit into the sample tubes (internal diameter=9.53 mm) were weighed and inserted into the sample tubes for degassing. Insertion of glass filler rods into the sample tubes was done to aid reduction of degassing time by reducing the total free space volume. The degassing conditions were set up comprising the evacuation and heating phases; and the parameters used are shown in Table 5. After about 21 hours of degassing, the sample tube was transferred to the analysis port for determination of surface area, pore size and volume in accordance to BET and BJH analysis. The analysis took about 5 hours and the analysis conditions are shown in Table 6.

TABLE 5
Degassing parameters for evacuation and heating phases

| Parameters | Target/Rate | |
|---|---|---|
| Evacuation phase | | |
| Temperature ramp rate | 10.0° | C./min |
| Target temperature | 30° | |
| Evacuation rate | 50.0 | mmHg/s |
| Unrestricted evacuation from | 30.0 | mmHg |
| Vacuum set point | 500 | μmHg |
| Evacuation Time | 60 | min |
| Heating Phase | | |
| Ramp rate | 10.0° | C./min |
| Hold temperature | 40° | C. |
| Hold time | 1320 | min |
| Hold pressure for evacuation and heating phases | 100 | mmHg |

TABLE 6
Parameter Settings for analysis conditions

| Features | Settings | |
|---|---|---|
| Preparations | | |
| Fast Evacuation | No | |
| Unrestricted evacuation from | 5.0 | mmHg |
| Vacuum setpoint | 10 | μmHg |
| Evacuation time | 0.10 | hour |
| Dosing | | |
| Use of first pressure fixed dose | No | |
| Use of Maximum volume increment | No | |
| Target tolerance | 5.0% or 5.0 mmHg | |
| Low pressure dosing | No | |
| Equlilbrium | | |
| Equilibrium time (P/Po = 1.0) | 20 | secs |
| Minimum equilibrium delay at P/Po >= 0.995 | 600 | secs |
| Sample backfill | | |
| Backfill at start of analysis | Yes | |
| Backfill at end of analysis | Yes | |
| Backfill gas | Nitrogen | |
| Adsorptive properties | | |
| Adsorptive | Nitrogen | |
| Maximum manifold pressure | 925.0 | mmHg |
| Non-ideality factor | 0.0000620 | |
| Density conversion factor | 0.0015468 | |
| Therm. Tran. Hard-sphere | 0.3860 | nm |
| Molecular cross-sectional area | 0.162 | nm² |

### Gastro-Adhesivity Testing of the Matrices

Freshly excised stomach tissue from a terminated pig was obtained and equilibrated in 0.1N HCl. The gastro-adhesive strength was determined using a Texture Analyzer (TA.XT plus, Stable Microsystems, UK). The parameters settings are shown in Table 7. The data was captured through Texture Exponent Software (V3.2). The peak force and the work of adhesion were used to assess the gastro-adhesivity of the matrices. The peak force is the maximum force required to detach the tissue from the matrices while the work of adhesion was determined from the Force-Distance profile.

TABLE 7
Parameter settings for the gastro-adhesivity test of the matrices

| Parameters | Settings |
|---|---|
| Pre test speed | 2 mm/sec |
| Test speed | 0.5 mm/sec |
| Post test speed | 10 mm/sec |
| Applied force¹ | 1 N or 0.5 N |
| Trigger type | Auto |
| Trigger force | 0.05 N |
| Contact time | 5 sec |
| Return distance | 20 mm |

### Determination of the Swelling of the Matrices

The swelling of the matrices was undertaken in 0.1N HCl. The matrices were weighed, placed in wire baskets and submerged in 100 mL of the medium and placed in a shaker bath (Orbital Shaker incubator, LM-530, Laboratory and Scientific Equipment Co, South Africa) at 37° C. Increase in mass was determined gravimetrically at time intervals over 24 hours. The degree of swelling was determined using Equation 3.

$$Degree of swelling = \frac{Wt - Wo}{Wo} \qquad Equation 3$$

where Wt is the weight of the matrix at time t, and Wo is the weight of matrix at time zero.

### In Vitro Drug Release Studies

Drug release was assessed using a USP 32 apparatus II dissolution system (Erweka DT 700, Erweka GmbH, Heusenstamm, Germany). Temperature and stirring rate was at 37±0.5° C. and 50 rpm respectively while the dissolution media was 0.1N HCl, buffers pH 1.5 and 4.5. Samples were withdrawn at predetermined intervals and replaced with the same volume of fresh medium, and the quantity of L-dopa released was quantified using UV spectroscopy. In vitro drug release studies were also undertaken for E3 matrices formulated from IPBs in varying normalities of acetic acid in buffer pH 1.5 (standard buffer KCl/HCl), pH 4.5 (0.025M KH₂PO₄/H₂PO₄) and pH 6.8 (standard buffer KH₂PO₄/NaOH) was employed to observe the behavior of the matrices and not for drug release as the model drug L-dopa was unstable at such pH values.

### Comparative In Vitro Drug Release Studies and Analytical Method

Comparative in vitro drug release study was undertaken with USP apparatus II dissolution system (Erweka DT 700, Erweka GmbH, Heusenstamm, Germany) at 37±0.5° C. and 50 rpm in 900 mL of buffer pH 1.5 for IPB, and the conventional products—Sinemet® CR and Madopar® HBS. Samples were withdrawn at time intervals over 24 hours. The same volume of fresh medium was added to each vessel after every withdrawal to maintain sink conditions and the concentrations of L-dopa, benserazide and carbidopa were quantified using Acquity™ Ultra Performance Liquid Chromatography (UPLC, Waters®, Manchester, UK) with methyl-dopa as internal standard. A gradient method was employed with mobile phase as water and acetonitrile running at 98% A (water), 0.50 min at 95% A, 0.70 min at 5% A and 95% at 1.00 min at a flow rate of 0.500 mL/min. Run time for L-dopa/Benserazide was 1.00 min and 1.20 min for L-dopa/Carbidopa. The column was Acquity UPLC® BEH shield RP18 1.7 μm, 2.1×100 mm. The wavelength employed was 210 nm, injection volume was 1.2 μL and temperature was 25° C.

Incorporation of the Polymer-Lipid Nanoparticles into the Interpolymeric Blend

The incorporation of polymer-lipid nanoparticles into the IPB was undertaken as described earlier via direct compression. However, the L-dopa-loaded polymer-lipid nanoparticles was incorporated instead of L-dopa alone while the in vitro drug release was assessed as described earlier. Typical compositions of nanoparticles utilized for incorporation into the IPB are shown in Table 8.

TABLE 8
Composition of the polymer-lipid nanoparticles

| | EUD | Chitosan | Levodopa | | |
|---|---|---|---|---|---|
| Formulation | (mg) | (mg) | (mg) | Lecithin (mL) | TPP (mg) |
| A22 | 150 | 150 | 100 | 1.00 | 250 |
| B3 | 150 | 50 | 100 | 1.00 | 50 |
| B6 | 100 | 100 | 100 | 1.00 | 50 |
| B9 | 200 | — | 100 | 1.00 | 50 |
| B12 | 50 | 50 | 100 | 1.00 | 100 |

### Results and Discussion

### Preparation of the Polymer-Lipid Nanoparticles

White EUD nanoparticles and creamy EUD/CHT were formed in the presence of lecithin and TPP. Polymeric miscibility was observed between EUD and CHT which may be due to the fact that they are both cationic polymers and so no interactions were observed. However, the enhancement of the individual properties of the polymers is envisaged through blending. EUD was not as viscous as CHT and it was envisaged that encapsulation of L-dopa may be lower with EUD alone. Surface adsorption may be more with EUD alone leading to rapid release of L-dopa. However, blending was expected to modulate drug release from the nanoparticles. On addition of lecithin, a color change (colloidal dispersion) was observed indicating the presence of interactions between lecithin (phospholipids) and the polymeric solution. Lecithin is an anionic phospholipid and surfactant which crosslinks cationic EUD and EUD/CHT polymeric solutions to produce polymer-lipid nanoparticles. The addition of TPP increased the degree of crosslinking which in turn influenced rate of drug release from the polymer-lipid nanoparticles.

### Assessment of the Size and Surface Charge of the Polymer-Lipid Nanoparticles

The average particle sizes for the nanoparticles after the addition of lecithin ranged from 152 nm for EUD only to 321 nm for EUD/CHT blend while the zeta potential ranged from 15.8-43.3 mV. As the quantity of CHT increased, the particle size increased. Furthermore, as the degree of crosslinking increased by the addition of TPP, the particle size increased to 424 nm. The polydispersity index ranged from 0.19-0.61.

### Assessment of Chemical Structure Variations of the Polymer-Lipid Nanoparticles

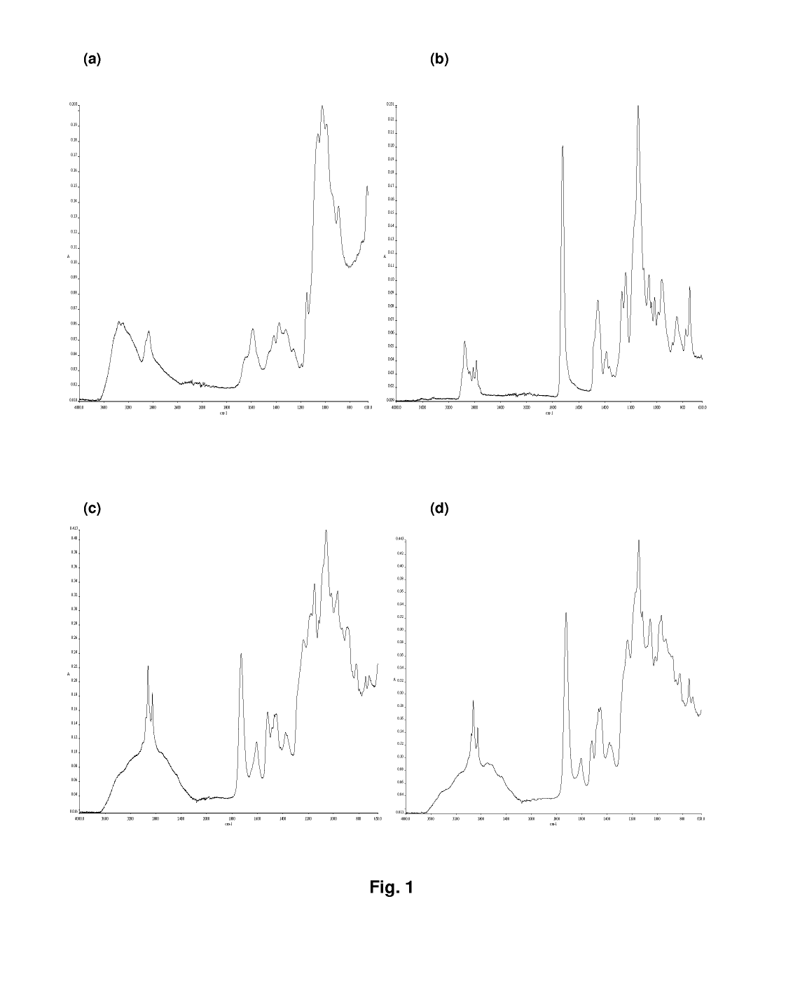

The FTIR spectra as shown in FIG. 1 exhibited chemical structural transitions that had occurred during nanofabrication by multi-crosslinking. In comparison with the spectra of the native polymers, the spectra of the nanoparticles showed the absence of some peaks found in the native polymers such as at 2769.74 cm⁻¹ and 1268.73 cm⁻¹ for EUD; 3357.51 cm⁻¹, 1590.66 cm⁻¹ and 1024.66 cm⁻¹ for CHT with the emergence of new peaks after crosslinking at 1605 cm⁻¹ which was found in EUD nanoparticles as well as the blend (EUD/CHT); 1519 cm⁻¹ in EUD that was slightly shifted in the blend to 1518.75-1522.24 cm⁻¹ envisaged to be determined by the degree of crosslinking in each nanoparticle formulation. Also the peaks in the native polymers which may be considered to still exist shifted slightly such as 2949.11 cm⁻¹ in EUD shifted to 2923.91 cm⁻¹, 1722.39 cm⁻¹ shifted to 1724.86 cm⁻¹ and 891.80 cm⁻¹ in CHT shifted to 889.79 cm⁻¹.

### Microscopical Analysis of the Levodopa-Loaded Poly-Lipo Nanoparticles

Scanning electron microscopy confirms the hollow capsules as envisaged and modelled (FIG. 2).

### In Silico Modelling, pH and Absorbance Changes During Fabrication of Poly-Lipo Nanoparticles

The chemical structure of methacrylate copolymer (Eudragit E100) possesses more room than chitosan for incoming entities and hence requires more TPP crosslinking. There is either of seven patterns the nanoparticle synthesis (with incoming entities-lecithin, levodopa and TPP incorporated into the polymeric matrix) may follow depending on the space, sizes of particles being formed initially and the presence or absence of turbulence. These patterns are tree branching, nodal space fillings, cone array formations, mixed triangular formations, linear patterns, chaotic patterns and mixed patterns. It is envisaged that the nanoparticle formation that occurred in this study may have been mixed triangle formation or mixed patterns. The description of the seven patterns has been discussed in a paper published by the inventors (Ngwuluka et al, 2011). Also in the publication is the Static Lattice Atomistic Simulations (in Silico) for prediction of the interaction mechanisms that occurred during synthesis of poly-lipo nanoparticles. Lecithin is an anionic phospholipid and surfactant that crosslinks with cationic methacrylate copolymer or methacrylate copolymer/chitosan polymeric solutions by electrostatic interactions to produce polymer-lipid (poly-lipo) nanoparticles. Other studies have confirmed the interactions between chitosan and phospholipids (lecithin) (Grant et al. 2005, Hafner et al. 2009, Ho et al. 2005, Lim Soo et al. 2008, Sonvico et al. 2006, Zahedi et al. 2009), while the interaction between methacrylate copolymer and lecithin was observed in this study. The functions of sequestration and crosslinking of TPP further binds the components in a nanoparticulate complex. The addition of TPP increased the degree of crosslinking which in turn influenced rate of drug release from the poly-lipo nanoparticles. Increase in concentration of polymers and TPP increased the pH of the nanosuspensions (Table 9). For the polymethacrylate copolymer/chitosan blend, pH increased as more components are added. However, increase in pH was more pronounced when TPP was added. Furthermore, with methacrylate copolymer alone—B9, there was no change in pH from the addition of L-dopa to that of lecithin.

TABLE 9
Comparative pH changes during nano-fabrication

| | | Addition | | Polymer + |
|---|---|---|---|---|
| Formulation | Polymer | of | Polymer + | L-dopa + |
| Code | Solution | L-dopa | L-Dopa + Lecithin | Lecithin + TPP |
| A22 | 1.17 | 1.31 | 1.36 | 3.15 |
| B3 | 1.17 | 1.34 | 1.40 | 1.73 |
| B6 | 1.18 | 1.36 | 1.41 | 1.78 |
| B9 | 1.13 | 1.28 | 1.28 | 1.68 |
| B12 | 1.14 | 1.19 | 1.23 | 1.78 |

pH of 0.2N HCL was 1.00.

On addition of lecithin to polymeric solutions, a color change (colloidal dispersion) was observed indicating possible interactions between lecithin (phospholipids) and the polymeric solution. It is also envisaged the color change could be due to the formation of capsular wall or surfactant activity. Furthermore, the color change may be depicting energy perturbation which was corroborated by in silico modeling. The oxygen excitation produces the color change-protons are absorbed while the rest of the visible spectrum wavelength is reflected back. The addition of TPP to the blended polymeric solutions (methacrylate copolymer and chitosan) gave a creamer color because of the oxygen-related functions (excitable oxygen atoms, conjugated oxygen containing groups in higher degree are present in chitosan and TPP). The intensity of visible light as indicated by absorbance increases as lecithin and TPP are added to polymeric solutions (Table 10) which is also an indication of color change and subsequent interactions between polymeric solution and the ionic agents (lecithin and TPP). However, it is observed that addition of TPP to methacrylate copolymer-lecithin blend led to decrease in absorbance. This is attributed to the chemical infrastructure of methacrylate copolymer which requires a higher quantity of TPP than utilized to achieve sufficient particulate complexation.

TABLE 10
Changes in absorbances during nano-fabrication

| Polymer | | Addition of | |
|---|---|---|---|
| Composition | Polymer Solution | Lecithin | Addition of TPP |
| EE100 | 0.0135 | 0.5681 | 0.4876 |
| Chitosan | 0.1382 | 3.3501 | 3.5597 |
| EE100 + Chitosan | 0.0589 | 2.7885 | 3.1930 |

EE100-methacrylate copolymer

### Surface Morphology of the Polymer-Lipid Nanoparticles

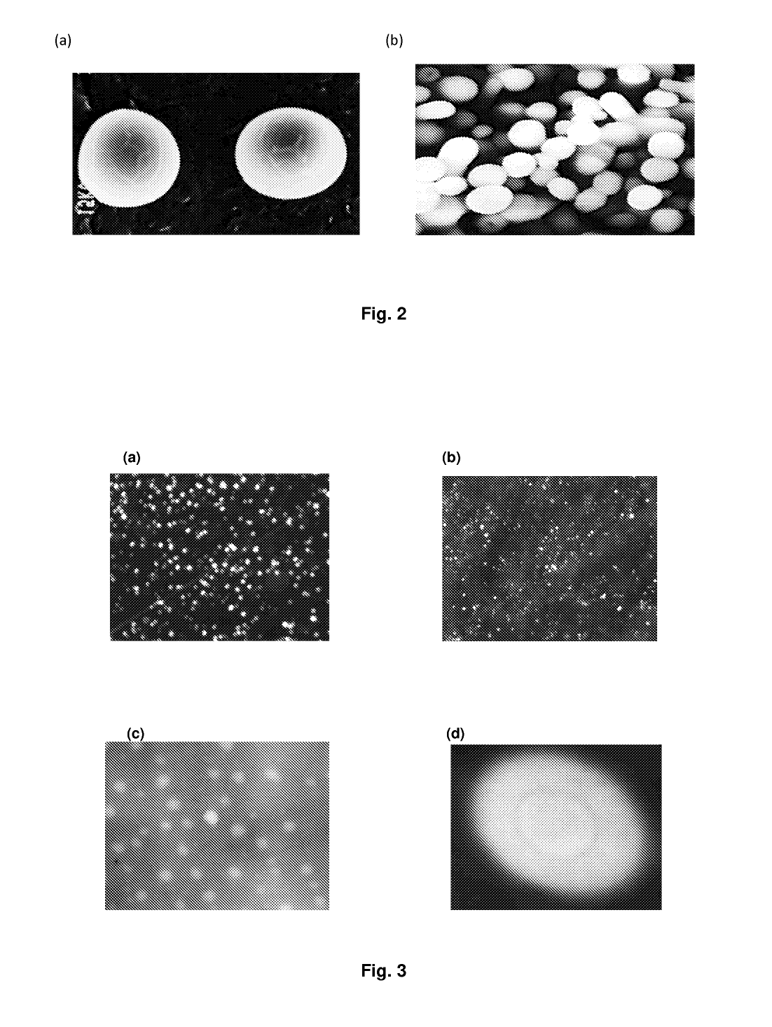

Spherical structured nanoparticles were observed when viewed under a digital microscope and TEM before lyophilization. FIG. 3 shows digital images of EUD/CHT crosslinked with lecithin only and multi-crosslinked EUD nanoparticles.

The smaller size of the EUD nanoparticles compared to the blend with CHT was further confirmed by the digital images. The TEM images further confirmed the spherical nature of the particles as well as indicating that the particles are nanocapsular with the magnified (×20000) TEM image showing the inner and outer cores.

### Surface Morphological Analysis of IPB Matrices

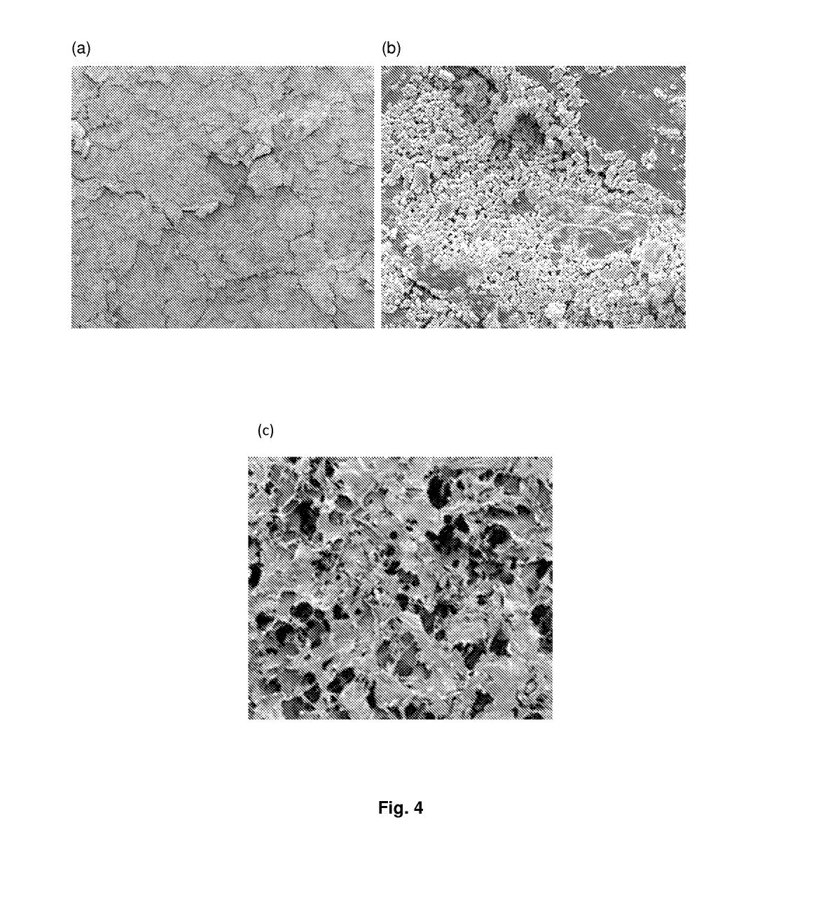

The Quanta™ Scanning Electron microscopical images of the non-hydrated and hydrated IPB polymer matrix are shown in FIGS. 4a, b and c. The pores are not visible in non-hydrated matrices. Pores are created by solvent penetration and drug dissolution making them visible. As the dissolution medium or buffer fills the initial voids in the matrix, L-dopa dissolves and diffuses out through the pores created by penetration of the solvent into the matrix. It is envisaged that creation of pores also involves the dissolution of other components such as pullulan. The microscopical image in FIG. 4c confirms that IPB matrices are porous swellable release systems. Amongst other mechanisms, pores contribute to the diffusion and diffusion-controlled mechanism of the release of L-dopa from the matrices. Pores as shown in FIG. 4c are not uniform and in addition, the release of L-dopa from the matrices can be attributed to drug dissolution and diffusion through the pores as well as swelling of the matrices.

### Porositometric Analyses of IPB Matrices

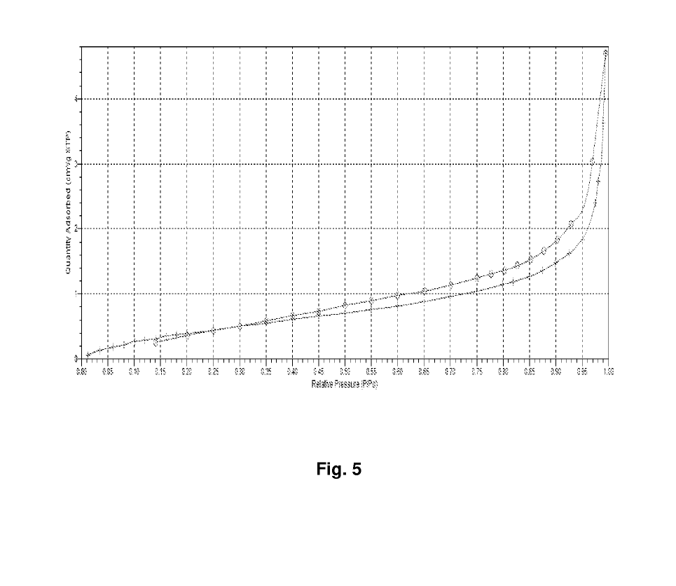

FIG. 5 shows a linear isothermic plot obtained, characteristic of physisorption isotherm Type IV with its hysteresis loop (probably H2) associated with capillary condensation that usually occur in mesopores. The forced closure (Tensile strength effect) of adsorption and desorption isotherms occurred in the P/Po range of 0.30 to 0.35 due to a sudden drop in the volume adsorbed along the desorption branch. Table 11 is a summary of the result obtained which corroborates the linear isotherm plot indicating that IPB matrices are mainly mesopores. About 92% of the pores are mesopores. The absence of micropores was confirmed by t-plot; though not used to determine micropore size but gives information on micropore volume. The micropore volume of IPB was negative (−0.000673 cm³/g) and as a result, the micropore area could not be determined. Hence, IPB matrices are mainly mesoporous indicating that one of the possible mechanisms of drug release from IPB is diffusion.

TABLE 11
A summary of surface area and pore analyses of IPB matrices

| Surface Area (m²/g) | | | | Pore Volume (cm³/g) | | | Ave Pore Size (nm) | | |
|---|---|---|---|---|---|---|---|---|---|
| SPSA | BET | BJH A | BJH D | SPAT | BJH A | BJH D | BET | BJH A | BJH D |
| 1.3640 | 1.9548 | 1.880 | 2.2217 | 0.0037 | 0.0071 | 0.0070 | 7.5762 | 15.1976 | 12.6376 |

SPSA—Single point surface area at P/Po = 0.200211845;
BJH A—BJH Adsorption cumulative surface area/volume of pores between 1.7 and 300 nm;
BJH D—BJH desorption cumulative surface area/volume of pores between 1.7 and 300 nm;
SPAT—Single point adsorption total pore volume of pores less than 78.9 nm diameter at P/Po = 0.9748.

### Drug-Loading Efficiency of the Polymer-Lipid Nanoparticles

The drug-loading efficiency was found to be 93%. The polymer-lipid nanoparticles had a high drug entrapment efficiency of 85%. Though the fabrication was stepwise there was no washing, centrifuging or decanting. It is envisaged that drug incorporation into the nanoparticles is a combination of encapsulation and surface adsorption.

### Synthesis of the Interpolymeric Blend

On addition of transparent EUD to a CMC solution, white strands were observed within the CMC gel for combination ratios of 1:0.5 and 1:1 of EUD and CMC respectively indicating incomplete interactions at such ratios. Hence at the end of 3 hours, the product appeared as an entangled gel with white strands. However at the ratio of 0.5:1 of EUD and CMC respectively, a homogenous white blend which was insoluble formed. At a 0.5:1 ratio, EUD, a cationic polymer and CMC, an anionic polymer interacted to form an inter-polyelectrolyte complex. The interactions involved in this complexation were strong ionic associations, hydrogen bondings and hydrophilic interactions. EUD interacted with acetate ions thereby stabilizing the ammonium cations of the polymer. As EUD was added to CMC, sodium acetate was generated that enhanced crosslinking between the two polymers. As agitation occurred, in the presence of water, acetic acid molecules and water held by hydrophilic interactions, sodium acetate was generated. For EUD and CMC to fully neutralize, excess CMC was required to generate sufficient salt for threshold crosslinking. A white insoluble inter-polyelectrolyte complex was formed at a ratio of 0.5:1 (EUD:CMC) which is distinct in a less viscous blend. The final viscosity of the inter-polyelectrolyte complex was dependent on the initial viscosity of CMC and the normality of acetic acid. As the normality of acetic acid shifted from 0.1-1.0N, the viscosity of the inter-polyelectrolyte complex decreased. There was no significant alteration of the blend observed with the addition of LB apart from an increase in viscosity. This was envisaged as LB is a neutral galactomannan polymer (Alves et al. 1999; Camacho et al. 2005; Sittikijyothin et al. 2005). The hydrophilic groups of LB associate with existing water molecules leading to a further increase in viscosity as the LB swells. The water molecules held within the IPB were sublimated during lyophilization resulting in a dry porous IPB. However, the degree of porosity increased with an increase in the normality of acetic acid.

### Analysis of the Chemical Structure Variation of the Interpolymeric Blend

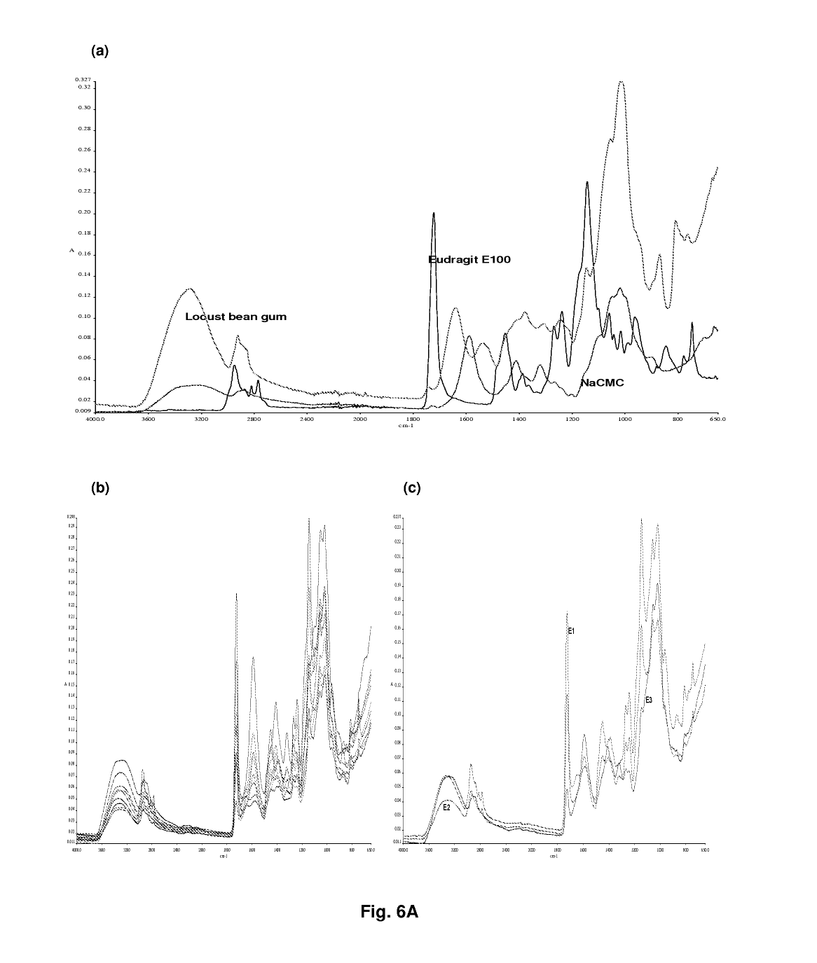

The spectra of the native polymers are shown in FIG. 6A(a) while the chemical structural transitions for the formulations are shown in FIGS. 6A(b-c) and 6B(d-e).

The characteristic peaks for EUD were found at 2821.42 cm⁻¹, 2769.84 cm⁻¹, 1725 cm⁻¹, 1270.38 cm⁻¹, 1239.56 cm⁻¹, 1143.69 cm⁻¹, 962.05 cm⁻¹, 842.49 cm⁻¹ and 747.81 cm⁻¹ while that of CMC were present at 3210.04 cm⁻¹, 1587.18 cm⁻¹, 1411.77 cm⁻¹, 1321.86 cm⁻¹ and 1019.59 cm⁻¹. The blend between EUD and CMC was a chemical interaction while incorporation of LB was envisaged to be a physical interaction. The chemical interactions between EUD and CMC led to the disappearance or diminished characteristic peaks of EUD at the homogenous ratio of 0.5:1 as seen in Formulation E3. The aliphatic aldehyde peaks of EUD at 2821.42 cm⁻¹ and 2769.84 cm⁻¹ had disappeared in Formulation E3 but was still present in Formulations E1 (ratio 1:0.5) and E2 (1:1). The other formulations were based on the same ratios 1:0.5, 1:1, 0.5:1 of EUD:CMC respectively. Hence the focus will be on the first three, E1, E2, and E3. The peak of EUD at 747 cm⁻¹ present in E1 and E2 disappeared in E3. However, the distinct carbonyl peak at 1725 cm⁻¹ diminished in E3 while it was still pronounced in E2 and E3. This may indicate that a few of the carbonyl groups may have been involved in the interaction while the aliphatic aldehyde groups may have been converted to aliphatic alcohols which would have sublimated during lyophilization. The peak at 1143.69 cm⁻¹ of EUD shifted insignificantly to 1145.59 cm⁻¹ but remained distinct in E1 and E2 while in E3 it appeared as a shoulder peak to the characteristic peak of CMC at 1019.12 cm⁻¹ which also shifted from 1019.59 cmcm⁻¹. In FIG. 6b, the blue spectrum is E10 which has a higher concentration of CMC, hence the characteristic peaks of CMC was more pronounced at 1587.18 cm⁻¹, 1411.77 cm⁻¹, 1321.86 cm⁻¹ and 1019.59 cm⁻¹. The impact of LB on the chemical structural modification could not be seen from the spectra except that of E1 which had a peak at 868.06 cm⁻¹ that was characteristic to LB. This is also due to the fact that E1 is more of a heterogeneous blend. Furthermore, it was envisaged that the homogeneity of E3 resulted in an almost superimposed spectra (FIG. 6e) with slight differences in the degree of absorbance at the various frequencies or peaks with E3 in 1.0N acetic acid having the highest degree of absorbance at peaks 1725 cm⁻¹, 1589 cm⁻¹, 1408 cm⁻¹, 1268.50 cm⁻¹ and 1019 cm⁻¹. However, E1 spectra were not superimposed, as the differences in degree of absorbance for each spectrum were distinct.

Direct Compression of the Interpolymeric Blend into Matrices

The IPB was directly compressible and not friable indicating that it would not require excipients to enhance compactness. Excipients added in this study were a density enhancing agent (BaSO₄), a glidant (silica) and a lubricant (magnesium stearate) to improve its flow properties and pullulan was used a bioadhesive agent. Direct compression is cost effective as it requires less excipients and steps of operations. It is suitable for drugs with stability challenges such as L-dopa which is moisture sensitive. In fact it is regarded as the tabletting method of choice for thermolabile and moisture sensitive drugs (Jivraj, et al. 2000). The IPB displayed excellent compatibility at 2 and 3 tons of compression with no evidence of friability, capping or lamination and it was found to be compatible with the model drug L-dopa.

### Assessment of the Density of the Matrices

The difference between the densities of the matrices from each formulation as shown in Table 12 was not significant. The densities ranged between 1.43 and 1.54 g/cm³. The densities obtained were indicative of the matrices' ability to sink down to the antrum of the stomach since they are significantly denser than the gastric contents of the stomach. Although density above 2.4 g/cm³ is advocated for high density delivery systems to ensure prolonged gastric residence time, it is envisaged the IPB matrices will still provide gastric residence with lower density than recommended since they are employing three approaches of gastroretention i.e., high density, swellability and gastro-adhesivity. From previous physiological studies it can be stated that non-disintegrating single unit drug delivery systems would remain in the stomach in the fed phase and would be emptied with the housekeeping wave (Davis et al. 1986). Drug delivery systems are more prone to clear from the stomach at fasted state than fed state due to housekeeping waves. Hence an IPB matrix with a density of 1.4 g/cm³ and non-disintegrating at gastric pH when ingested will sink to the antrum of the stomach and will only be emptied during housekeeping waves. Furthermore to ensure prolonged gastric residence time, it may be taken during the fed state.

TABLE 12
Density results obtained for the various IPB matrices

| Formulation | Density (mg/mm³ or g/cm³) |
|---|---|
| E1 | 1.51 |
| E2 | 1.54 |
| E3 | 1.50 |
| E4 | 1.54 |
| E5 | 1.50 |
| E6 | 1.50 |
| E7 | 1.51 |
| E8 | 1.50 |
| E9 | 1.52 |
| E10 | 1.51 |
| E1 0.2N | 1.52 |
| E1 0.4N | 1.47 |
| E1 0.6N | 1.46 |
| E1 0.8N | 1.52 |
| E1 1.0N | 1.50 |
| E3 0.2N | 1.45 |
| E3 0.4N | 1.43 |
| E3 0.6N | 1.47 |
| E3 0.8N | 1.48 |
| E3 1.0N | 1.50 |

### Physicomechanical Strength Analyses of the Matrices

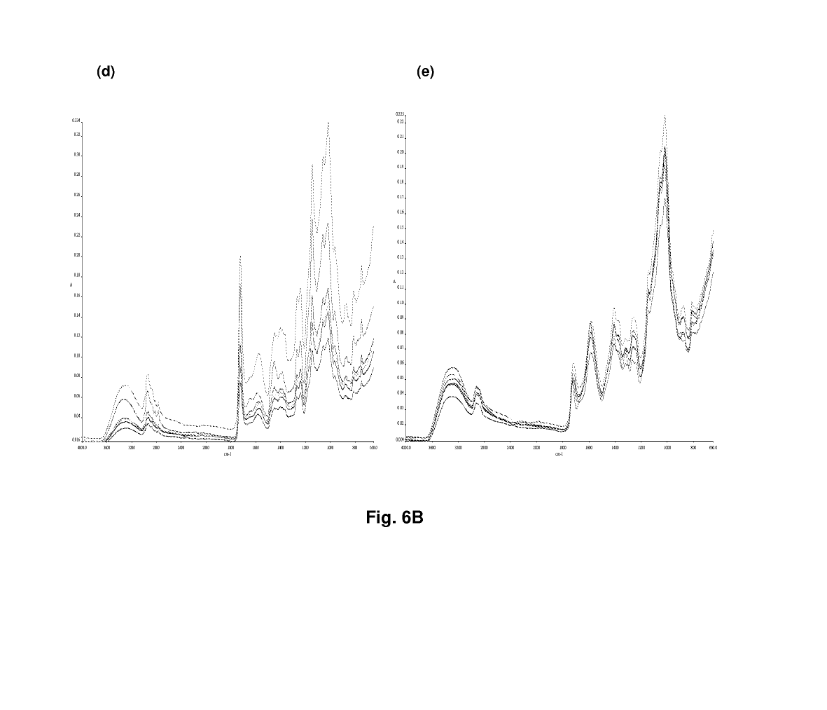

Physicomechanical strength analysis was undertaken since the Matrix Hardness (MH) and Matrix Resilience (MR) are an indication of the stability of the matrices and their ability to withstand pressure during compression and its capability to restore to its original dimensions after the compressional stress applied during textural analysis. MR also influences the drug release kinetics. MH and MR indicates the degree of density and porosity of a matrix which affects the drug release profile from the matrix by affecting the rate of penetration of the dissolution medium into the matrix (Nur, 2000). Less MH and MR may indicate the presence of voids which collapse on application of stress. Porosity also determines the quantity of deformation energy required; the harder the matrix, the less the energy absorbed or the more the deformation energy which also affect the MR. The inherent properties of the polymers utilized in formulation of the matrices also determine the degree of MH. In this study, it was also observed that lyophilization could also strengthen the physicomechanical properties of polymers causing native polymers to retain their three dimensional networks. The different formulations as shown in Table 13 indicated superior MH that ranged from 34.720-39.707N/mm; the deformation energy ranged from 0.012-0.014 Nm while the MR ranged from 44.25-47.65%. Hence all formulations had superior physicomechanical strength and would be able to withstand processing stressors. Typical Force-Distance and Force-Time profiles obtained are shown in FIG. 7. FIG. 7a indicates matrix hardness and deformation energy and FIG. 7b indicates matrix resilience of the IPBs.

TABLE 13
Texture profiling results of the various IPB formulations

| | Matrix Hardness | Deformation | Matrix |
|---|---|---|---|
| Formulation | (N/mm) | Energy (Nm) | Resilience (%) |
| E1 | 39.364 | 0.012 | 45.39 |
| E2 | 38.419 | 0.012 | 44.25 |
| E3 | 38.919 | 0.012 | 46.68 |
| E4 | 38.897 | 0.012 | 46.23 |
| E5 | 39.707 | 0.012 | 46.52 |
| E6 | 38.367 | 0.012 | 46.86 |
| E7 | 37.042 | 0.012 | 46.79 |
| E8 | 37.07 | 0.012 | 47.65 |
| E9 | 38.403 | 0.012 | 45.43 |
| E10 | 35.769 | 0.013 | 47.65 |
| E1 0.2N | 37.317 | 0.012 | 46.75 |
| E1 0.4N | 37.961 | 0.013 | 47.22 |
| E1 0.6N | 36.497 | 0.013 | 46.15 |
| E1 0.8N | 36.316 | 0.013 | 46.80 |
| E1 1.0N | 36.683 | 0.013 | 46.37 |
| E3 0.2N | 35.349 | 0.013 | 46.25 |
| E3 0.4N | 34.72 | 0.013 | 46.36 |
| E3 0.6N | 34.937 | 0.013 | 46.72 |
| E3 0.8N | 35.027 | 0.014 | 45.98 |
| E3 1.0N | 36.393 | 0.013 | 46.32 |

### Polymeric Nanoparticles Improve Mechanical Strength of Matrices

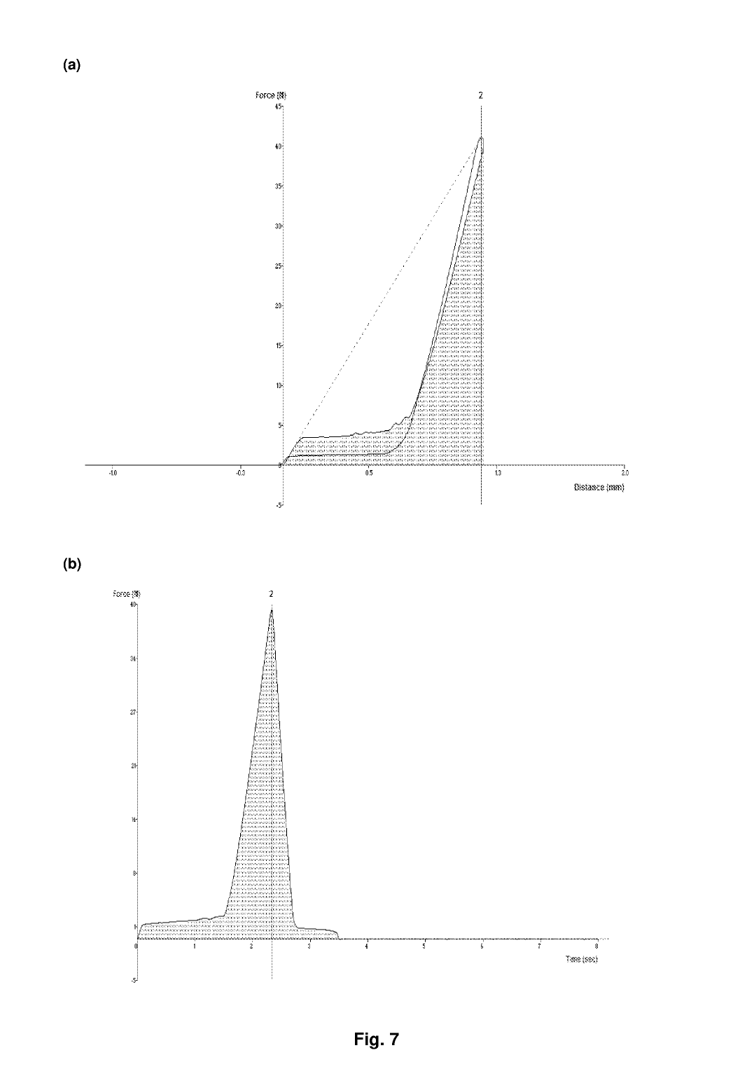

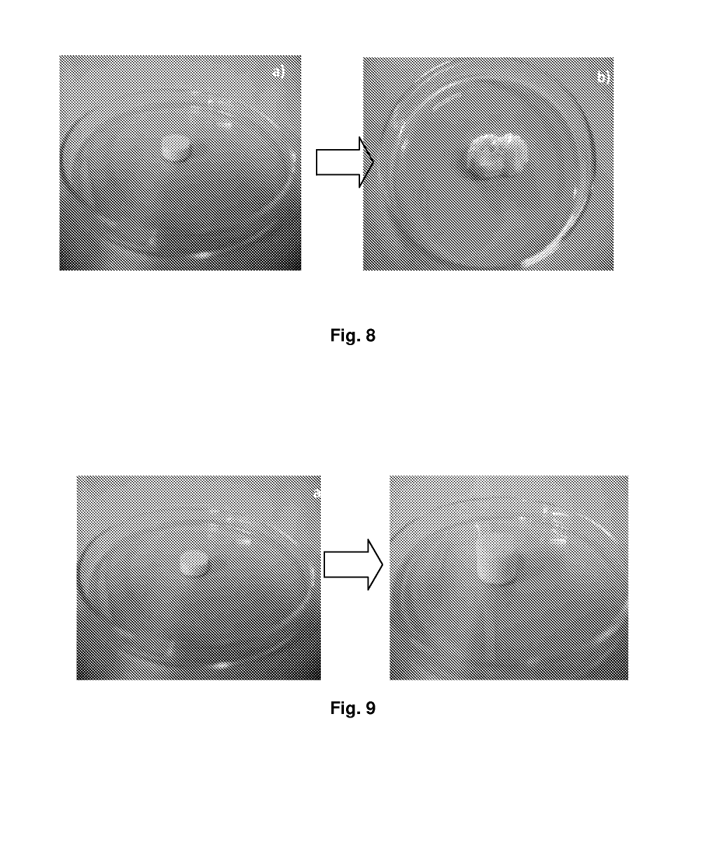

The interpolymeric blend is a pH responsive material which maintains its three-dimensional network in pH 1.5 but undergoes surface erosion in higher pH such as 4.5. However, when poly-lipo nanoparticles are incorporated into the polymeric blend and compressed, the three-dimensional network is maintained in both buffer types over the 24 h drug release studies. Studies have shown that nanoparticles can be employed to improve the mechanical strength of matrices (Beun et al. 2007, Gojny et al. 2005, Gomoll et al. 2008, Park, Jana 2003, Rapoport et al. 2004, Saha, Kabir & Jeelani 2008, Zhang et al. 2003). These studies used inorganic nanoparticles to enhance mechanical properties. However, in this study polymeric nanoparticles improved the mechanical strength of a polymeric matrix preventing the polymeric matrix's erosional response at a higher pH. The pictorial diagram of the impact of nanoparticles on the mechanical strength of the interpolymeric blend matrix is shown in FIGS. 8 and 9.

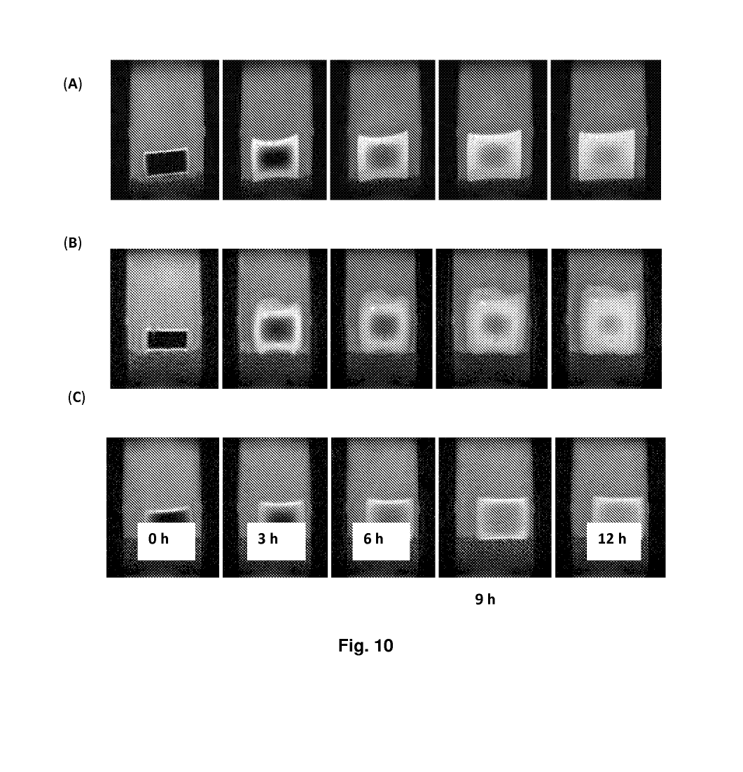

Magnetic resonance imaging was used to confirm the mechanical behaviors of interpolymeric blend in the absence and presence of poly-lipo nanoparticles. FIG. 10A shows images obtained at pH 1.5 when nanoparticles were incorporated into the polymeric blend. FIG. 10B shows the gradual erosion of the interpolymeric blend without nanoparticles at pH 4.5 while FIG. 10C displays the enhancement of the matrix upon incorporation of nanoparticles at pH 4.5. The images obtained at 0, 3, 6, 9 and 12 h are shown in FIG. 10. Surrounding the matrix is the dissolution medium (the grey part); the black portion within the tablet matrix is the non-hydrated part of the tablet and the white part indicates the hydrated, swollen and gelled portion. As the matrix hydrates, the thickness of the white portion increases over time until the matrix is fully hydrated. The presence of nanoparticles in the interpolymeric blend at pH 4.5 prevented surface erosion. Less penetration of solvent into the matrix was observed in FIG. 10C as the thickness of the white part was less as compared to images in FIG. 10A and hence, less swelling and gelling. Less water penetration is also partly due to the pH responsiveness of interpolymeric blend. It is envisaged that the presence of nanoparticles in the tablet matrix prevented erosion and retained the three-dimensional network of the matrix due to electrostatic interactions between the nanoparticles and the interpolymeric blend.

### Gastroadhesivity Testing of the Matrices

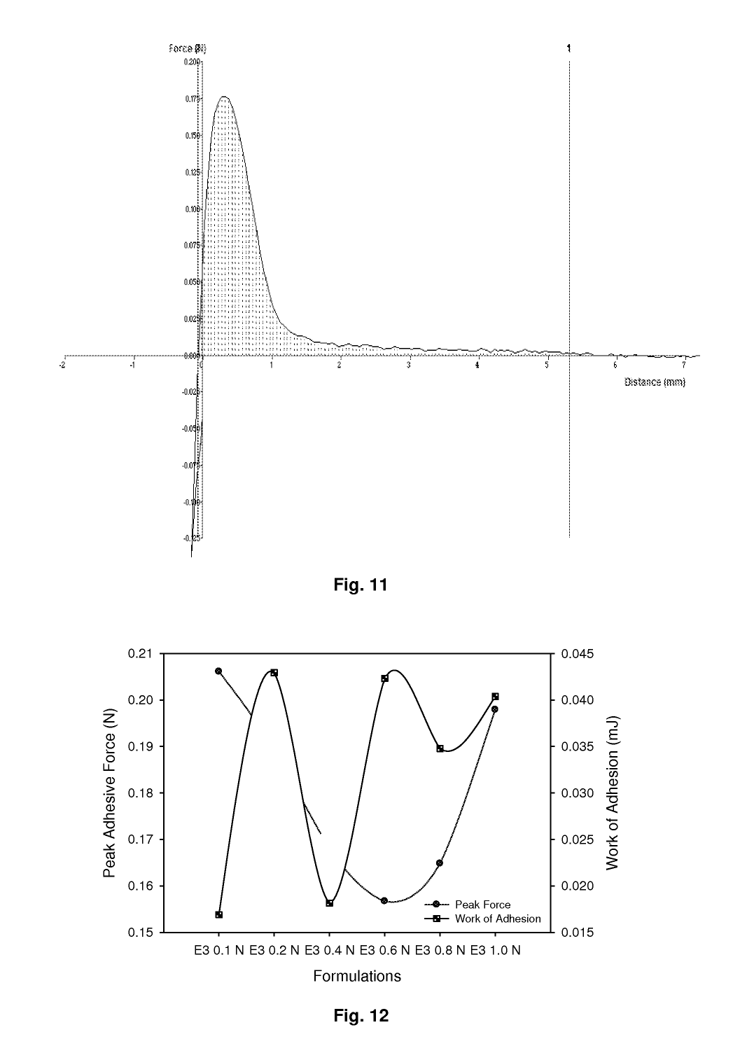

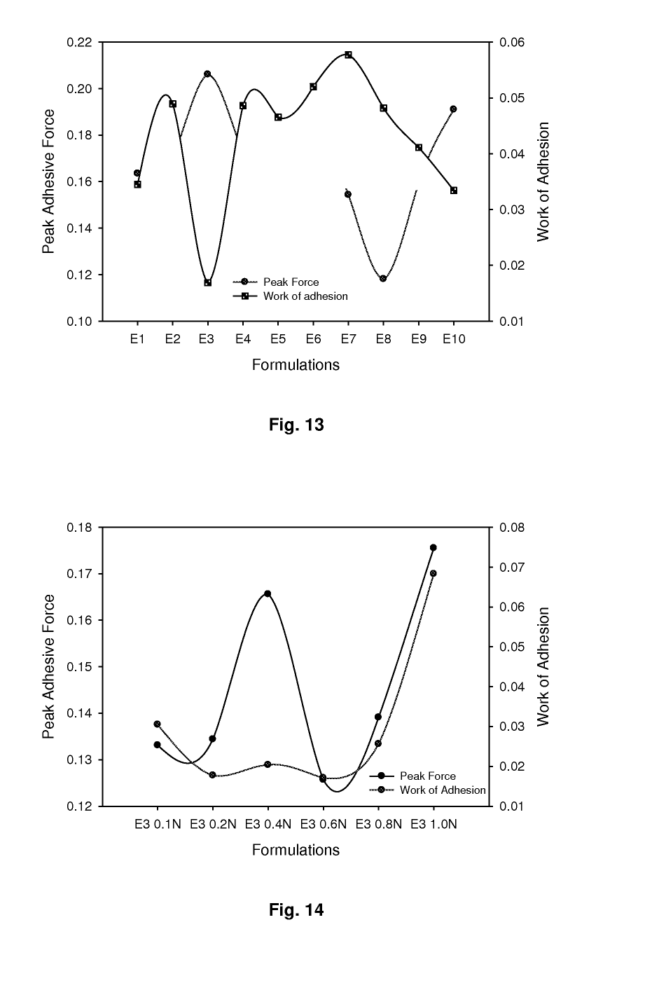

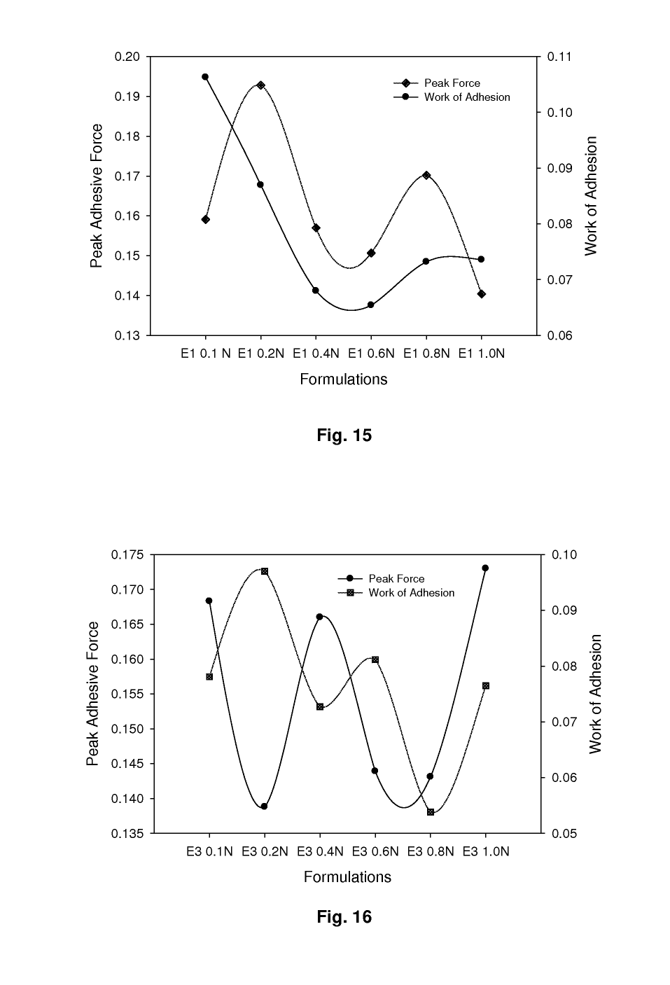

The IPB matrices of varying concentrations of polymers and normality's of acetic acid were found to be gastro-adhesive as shown in FIGS. 12-16 while FIG. 11 shows a typical gastro-adhesive Force-Distance profile obtained. The interactions between the gastric mucosal surfaces and drug delivery systems formulated from bioadhesive polymers include covalent bonding, hydrogen bonding, electrostatic forces such as Van der Waal forces, chain interlocking and hydrophobic interactions (Lee et al., 2000; Thirawong et al., 2008; Woodley 2001) and these interactions are regulated by pH and ionic conditions. The degree of interaction between the polymers and mucus is also dependent on the mucus viscosity, degree of entanglement and water content (Lee et al., 2000). As the applied force is increased from 0.5N to 1N, the peak adhesive force and work of adhesion increased. Increased applied force will increase intimate contact by causing viscoelastic deformation at the interface between the mucus and the drug delivery system (Lee et al., 2000). Although the contact time employed was 5 seconds, the gastro-adhesive results were commensurable for gastro-adhesive strength which will increase as contact time increases and subsequently increases the interpenetration of the polymeric chains. The peak adhesive force and work of adhesion was found to be higher when the IPB matrices adhered to the gastro epithelium. This may have been enhanced by the presence of a microbial adhesive agent, pullulan from Aureobasidium pullulans in the matrices. Microbial adhesions are postulated to have the capability to increase mucoadhesion to the epithelium (Vasir et al. 2003).

### Assessment of the Matrix Swelling

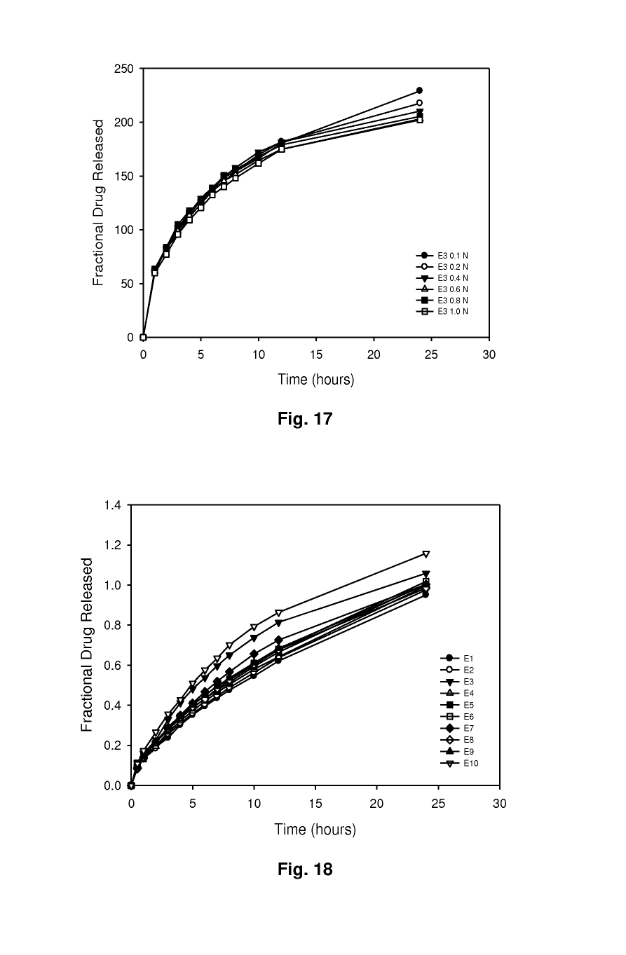

Drug release kinetics from a polymeric matrix are affected by structural features of the network, process of hydration, swelling and degradation of the polymer(s) (O'Brien et al., 2009). As the dissolution medium is absorbed by the matrix, this results in swelling and the incorporated drug dissolves and diffuses through the pores and out of the matrix. The rate of diffusion depends on the degree of swelling thereby affect the quantity of drug released with time. The swelling is affected by the polymer-solvent interaction, presence of drug and degree of crosslinking (Kim, Bae et al., 1992). Increasing the degree of crosslinking would lower the degree of swelling thereby reducing water content and subsequent diffusion of drug from the hydrogel (Wise, 1995). Matrices formulated with EUD alone dissolves in an acidic medium while CMC alone swells to 384% of its original size with loss of its three dimensional network. However the EUD/CMC blend formed swells much more than CMC. On addition of LB, its hydrophilic groups associate with the water holding capacity of the EUD/CMC blend thereby reducing the degree of swelling of the blend below 300%. Table 14 exhibits the degree of swelling of the various Formulations at t=24 hours. However, E3 was chosen to determine the degree of swelling at time intervals in the day. Formulation E3 was selected since the inter-polyelectrolyte complex was obtained at a ratio of (0.5 EUD:1.0 CMC) and FIG. 17 depicts the degree of swelling profile over 24 hours. It was observed that the degree of swelling decreased as the normality of acetic acid increased from 229% of 0.1N to 202% of 1.0N of acetic acid.

TABLE 14
Degree of swelling results obtained for the various IPB matrices

| Formulation | Degree of swelling (%) |
|---|---|
| E1 | 221.30 |
| E2 | 187.91 |
| E3 | 218.19 |
| E4 | 204.62 |
| E5 | 220.10 |
| E6 | 241.36 |
| E7 | 200.81 |
| E8 | 211.63 |
| E9 | 177.36 |
| E10 | 183.36 |
| E1 0.2N | 216.46 |
| E1 0.4N | 218.50 |
| E1 0.6N | 203.90 |
| E1 0.8N | 218.85 |
| E1 1.0N | 234.25 |

### In Vitro Drug Release Studies

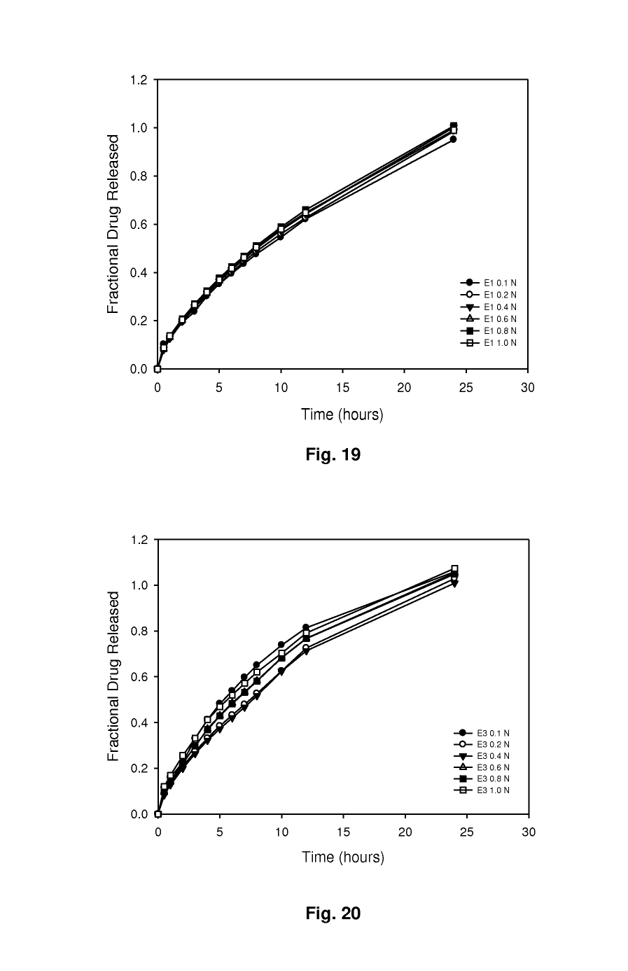

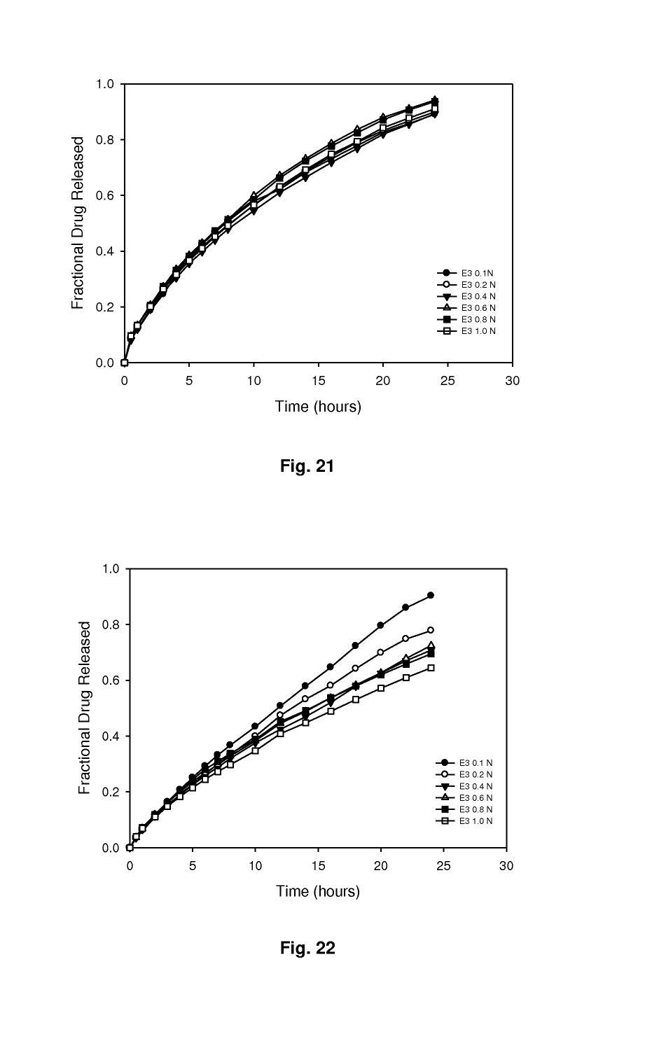

Drug release profiles were obtained and the three dimensional network of the matrices were retained over a 24 hour period. However, after dissolution on physical touch of the hydrated matrices, it was observed that E5 was the softest due to the higher concentration of EUD which was three times greater than the concentration of CMC with weak associations as more CMC was required for stronger interactions. Those that required a little pressure on touch to collapse were E3, E7, and E10 due to the presence of more CMC in the formulation than EUD. In FIG. 18, the drug release profiles of Formulations E3, E7 and E10 were distinct. The degree of crosslinking in the aligned profiles may have been little or none due to the weak interactions and minimal salt generation during synthesis of the IPB. The mechanism of drug release was clearly by swelling and diffusion since the matrices retained their three dimensional network. E1 was selected and synthesized in varying normality's of acetic acid. However, not much difference was observed as the profiles practically aligned with each other as depicted in FIG. 19. Although there were increased acetate ions as the normality increased, the required salt for threshold crosslinking was not generated due to the lower concentration of CMC. However, differences in drug release could be seen when E3 was chosen (FIG. 20). The differences in profiles indicate the varying degree of crosslinking with the varying normality's of acetic acid. The matrices in the dissolution media 0.1N HCl and buffer pH 1.5 (standard buffer KCl/HCl) generated the drug release profiles in FIGS. 20 and 21 respectively and still retained their three dimensional networks. Hence mechanisms of drug release involved in these media were swelling of the matrix, dissolution and then simultaneous diffusion of drug from the matrix. Interestingly, as the pH was increased to 4.5, the matrices swelled with time but there was gradual surface erosion throughout the 24 hour period indicating the pattern of drug release pattern from the IPB may be pH dependent. Consequently, the drug release profiles at pH 4.5 as shown in FIG. 22 differed from those obtained in pH 1.5 or 0.1N HCl. Surface erosion occurs when the rate of erosion is greater than the rate of hydration and swelling (rate of absorption of dissolution medium) of the matrix and occurs at constant velocity which leads to reproducible kinetics of erosion and drug release which is usually zero order (Pillai, 2001; Faisant et al., 2002; Burkersroda et al., 2002; Siepmann, 2001). Hence, the mechanism of drug release in pH 4.5 was principally surface erosion, then swelling of the matrix, dissolution and then subsequent diffusion of the drug from the matrix producing zero-order release kinetics. It was observed the matrices did not completely erode after 24 hours. However, the degree of erosion decreased as the normality of acetic acid increased which in turn affected the drug release profile as shown in FIG. 22. A more linear drug release profile (zero-order) profile was obtained for E3 in 0.1N acetic acid which eroded greater than the other formulations indicating that erosion may have been its principal mechanism of release. Although the dissolution was undertaken in pH 6.8, the focus was not on drug release but on the behavior of the matrices at a pH of 6.8. This is because the model drug used is unstable at pH 6.8 and therefore the percentage drug release was not obtained. However, it was observed that the matrices underwent surface erosion as well.

### Comparative In Vitro Drug Release Studies

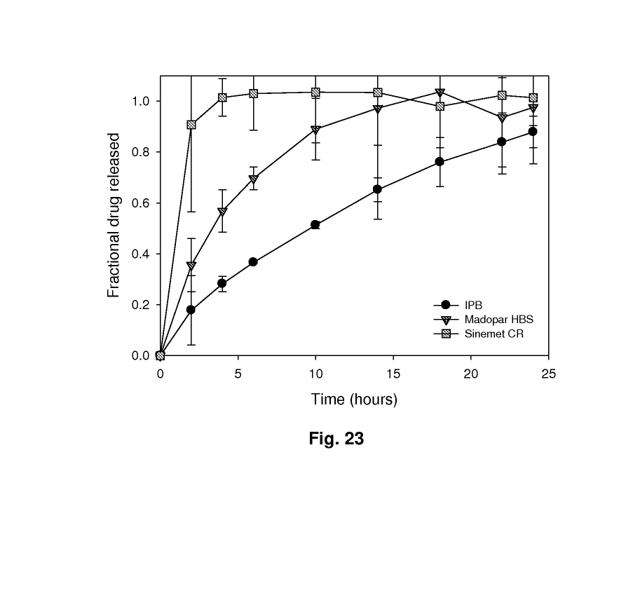

FIG. 23 shows the comparative drug release profiles of IPB matrices and conventional dosage forms—Madopar® HBS and Sinemet® CR. A more linear profile was obtained with IPB matrices. In comparison with the conventional dosage forms, interpolymeric blend shows promise as an oral delivery system that may improve the absorption and subsequent bioavailability of L-dopa/carbidopa with constant therapeutic plasma concentrations.

Density and In Vitro Drug Release from the Polymer-Lipid Nanoparticles Embedded in the Interpolymeric Blend

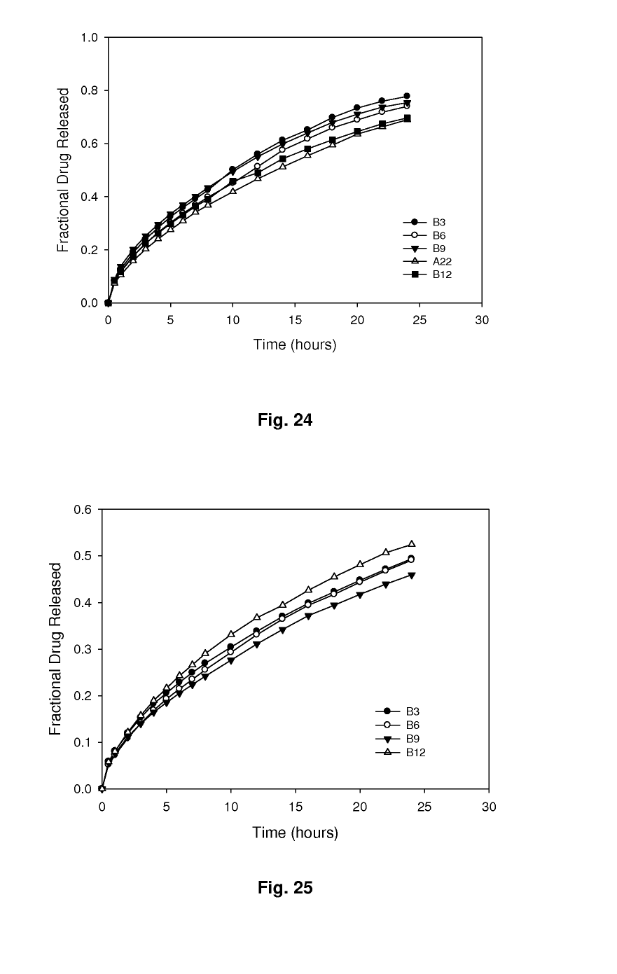

In comparison with drug release profiles of L-dopa-loaded IPBs, the L-dopa-loaded polymer-lipid nanoparticles embedded within the IPB matrix decreased the rate of drug release over a 24 hour period are illustrated in FIG. 24 and FIG. 25. The lowest fractional drug released in dissolution medium pH 1.5 from L-dopa-loaded IPBs was 0.8911 while that from L-dopa polymer-lipid nanoparticles embedded within the IPB matrix was 0.6896. Due to the decreased rate of hydration at pH 4.5, the lowest fractional drug released from L-dopa-loaded IPBs was 0.6445. However, the drug release from L-dopa-loaded polymer-lipid nanoparticles embedded within IPB matrices was much lower at pH 4.5. This was due to a further decreased rate of hydration and swelling of the IPBs due to the presence of the nanoparticles. It was also observed that the IPBs did not erode in the presence of the polymer-lipid nanoparticles at pH 4.5. It is envisaged that interactions between the nanoparticles and the IPB may have occurred at pH 4.5 preventing the surface erosion of the matrices. Hence in the absence of surface erosion, lower rates of hydration and swelling, approximately 50% of L-dopa was released from the L-dopa-loaded polymer-lipid nanoparticles embedded within the IPB matrices after 24 hours. However, it was also observed that the degree of crosslinking may have reduced the rate of L-dopa release. Furthermore improving the drug-loading efficiency by decreasing the quantities of polymers as well as crosslinking agents may increase the rate of drug release from the polymer-lipid nanoparticles within the IPB matrices.

### CONCLUSIONS

Multi-crosslinked polymer-lipid nanoparticles have been synthesized that are capable of high drug entrapment and able to modulate the rate of drug release. An inter-polyelectrolyte complex was formed at a stoichiometrical ratio of 0.5:1 (EUD:CMC). A triple mechanism gastroretentive drug delivery system has been designed and developed which has the potential to improve the absorption and bioavailability of narrow absorption drugs such as L-dopa. Furthermore, a polymer-lipid nanoparticulate enabled gastro-retentive matrix has been engineered which will be retained at the antrum of the stomach to facilitate continuous release and modulate the release of L-dopa at a constant and sustained rate over a prolonged period, enhancing the absorption and subsequent bioavailability thereby achieving an effective therapeutic outcome.

### REFERENCES

1. Adler, C. H. 2002, Relevance of motor complications in Parkinson's disease, Neurol. 58(90001), 51-56.
2. Alves, M. M., Antonov, Y. A. and Gonçalves, M. P., 1999. On the incompatibility of alkaline gelatin and locust bean gum in aqueous solution. Food Hydrocolloids, 13(1), 77-80.
3. Arza, R., Gonugunta, C. and Veerareddy, P., 2009. Formulation and evaluation of swellable and floating gastroretentive ciprofloxacin hydrochloride tablets. AAPS PharmSciTech, 10(1), 220-226.
4. Barone, P. 2003, Clinical strategies to prevent and delay motor complications, Neurol., 61, 12-16.
5. Birkmayer, W. and Hornykiewicz, 0., (1961) W. Birkmayer and O. Hornykiewicz, Der 1-3,4-dioxyphenylalanin (DOPA)-Effekt bei Parkinson-Akinese. Wien Klin Wochenschr 73, 787-788.
6. Brocks, D. R. 1999, “Anticholinergic drugs used in Parkinson's disease: An overlooked class of drugs from a pharmacokinetic perspective”, J. Pharm. Pharmaceut. Sci., 2(2), 39-46.
7. Burkersroda, F. V., Schedl, L. and Göpferich, A., 2002. Why degradable polymers undergo surface erosion or bulk erosion. Biomat., 23(21), 4221-4231.
8. Camacho, M. M., Martínez-Navarrete, N. and Chiralt, A., 2005. Rheological characterization of experimental dairy creams formulated with locust bean gum (LBG) and λ-carrageenan combinations. Int. Dairy J., 15(3), 243-248.
9. Davis, S. S., Hardy, J. G. and Fara, J. W., 1986. Transit of pharmaceutical dosage forms through the small intestine.
10. Descombes, S., Bonnet, A. M., Gasser, U. E., Thalamas, C., Dingemanse, J., Arnulf, I., Bareille, M. P., Agid, Y. & Rascol, O. 2001, “Dual-release formulation, a novel principle in L-dopa treatment of Parkinson's disease”, Neurol., 56(9), 1239-1242.
11. Ehringer H. and Hornykiewicz O., (1960). Verteilung von noradrenalin und dopamin (3-
12. hydroxytyramin) im gehirn des menschen und ihr verhalten bei erkrankungen. Wien. Klin. Wochenschr. 38, 1236-1239.
13. Fahn, S. 2008, “The history of dopamine and L-dopa in the treatment of Parkinson's disease” Movement Disord., 23(3), 497-508.
14. Faisant, N., Siepmann, J. and Benoit, J. P., 2002. PLGA-based microparticles: elucidation of mechanisms and a new, simple mathematical model quantifying drug release. Eur. J. Pharm. Sci., 15(4), 355-366.
15. Gasser, U. E., Crevoisier, C., Ouwerkerk, M., Lankhaar, G. & Dingemanse, J. 1998, “Comparative single- and multiple-dose pharmacokinetics of L-dopa and 3-O-methyldopa following a new dual-release and a conventional slow-release formulation of L-dopa and benserazide in healthy subjects”, Eur. J. Pharm. Biopharm., 46(2), 223-228.
16. Goole, J., Vanderbist, F. & Amighi, K. 2007, “Development and evaluation of new multiple-unit L-dopa sustained-release floating dosage forms”, Int. J. Pharm., 334(1-2), 35-41.
17. Gordon, M., Markham, J., Hartlein, J. M., Koller, J. M., Loftin, S. & Black, K. J. 2007, “Intravenous L-dopa administration in humans based on a two-compartment kinetic model”, J. Neurosci. Meth., 159(2), 300-307.
18. Hauser, R. A. 2004, “L-dopa/carbidopa/entacapone (stalevo)”, Neurol. 62, 564-571.
19. Hoffman, A., Stepensky, D., Lavy, E., Eyal, S., Klausner, E. and Friedman, M., 2004. Pharmacokinetic and pharmacodynamic aspects of gastroretentive dosage forms. Int. J. Pharm., 277(1-2), 141-153.
20. Hsu, A. F. & Han, C-H. 2006, “Oral disintegrating dosage forms”, Patent No. WO/2006/073729. Available online at http://www.wipo.int [Accessed: Oct. 5, 2008].
21. Iyer, S. S., Morgan, J. C. & Sethi, K. D. 2005, “Absorption of orally disintegrating carbidopa-L-dopa requires intact small bowel function”, Neurol., 65(9), 1507.
22. Jankovic, J. 2008, “Parkinson's disease: clinical features and diagnosis”, J. Neurol., Neurosurg. Psych., 79(4), 368-376.
23. Jivraj, M., Martini, L. G. and Thomson, C. M., 2000. An overview of the different excipients useful for the direct compression of tablets. Pharm. Sci. Tech Today, 3(2), 58-63.
24. Kim, S. W., Bae, Y. H. and Okano, T., 1992. Hydrogels: Swelling, drug loading, and release. Pharm. Res., 9(3), 283-290.
25. Klausner, E. A., Eyal, S., Lavy, E., Friedman, M. & Hoffman, A. 2003, “Novel L-dopa gastroretentive dosage form: in-vivo evaluation in dogs”, J. Control. Rel., 88(1), 117-126.
26. Koller, W. C., Hutton, J. T., Tolosa, E. & Capilldeo, R. 1999, “Immediate-release and controlled-release carbidopa/L-dopa in PD: A 5-year randomized multicenter study”, Neurol., 53(5), 1012.
27. Kordower, J. H. & Goetz, C. G. 1999, “The first miracle in neurodegenerative disease: The discovery of oral L-dopa”, Brain Res. Bulletin, 50(5-6), 377-378.
28. Lee, J. W., Park, J. H. and Robinson, J. R., 2000. Bioadhesive-based dosage forms: The next generation. J. Pharm. Sci., 89(7), 850-866.
29. Muzzi, C., Bertocci, E., Terzuoli, L., Porcelli, B., Clari, I., Pagani, R & Guerranti, R. 2008, “Simultaneous determination of serum concentrations of L-dopa, dopamine, 3-O-methyldopa and α-methyldopa by HPLC”, Biomed. Pharmacother. 62, 253-258.
30. Nur, A. and Zhang, J., 2000. Captopril Floating and/or Bioadhesive Tablets: Design and Release Kinetics. Drug Devel. Indus. Pharm., 26(9), 965.
31. Obering, C. D., Chen, J. J. & Swope, D. M. 2006, “Update on Apomorphine for the Rapid Treatment of Hypomobility (“Off”) Episodes in Parkinson's Disease”, Pharmacother., 26(6), 840-852.
32. O'brien, S., Wang, Y., Vervaet, C. and Remon, J. P., 2009. Starch phosphates prepared by reactive extrusion as a sustained release agent. Carb. Polym., 76(4), 557-566.
33. Olanow, C. W. & Stocchi, F. 2004, “COMT inhibitors in Parkinson's disease: Can they prevent and/or reverse L-dopa-induced motor complications?”, Neurol., 62(90011), 72-81.
34. Périer, C., Marin, C., Jimenez, A., Bonastre, M. & Tolosa, E. and Hirsch, E. C. 2003, “Effect of subthalamic nucleus or entopeduncular nucleus lesion on L-dopa-induced neurochemical changes within the basal ganglia and on L-dopa-induced motor alterations in 6-hydroxydopamine-lesioned rats”, J. Neurochem., 86(6), 1328-1337.
35. Pillai, O. and Panchagnula, R., 2001. Polymers in drug delivery. Curr. Opin. Chem. Biol., 5(4), 447-451.
36. Rouge, N., Buri, P. and Doelker, E., 1996. Drug absorption sites in the gastrointestinal tract and dosage forms for site-specific delivery. Int. J. Pharm., 136(1-2), 117-139.
37. Rubin, A. A. 2000, “Improvement in the treatment of Parkinson's disease and related disorders by novel formulations of the combination carbidopa-L-dopa” Patent No. WO/2000/015197. Available online at http://www.wipo.int [Accessed: Oct. 22, 2008].
38. Schapira, A. H. V. 2005, “Present and future drug treatment for Parkinson's disease”, J. Neurol. Neurosurg. Psych., 76(11), 1472-1478.
39. Siepmann, J. and Göpferich, A., 2001. Mathematical modeling of bioerodible, polymeric drug delivery systems. Adv. Drug Deliv. Rev., 48(2-3), 229-247.
40. Sittikijyothin, W., Torres, D. and Gonçalves, M. P., 2005. Modelling the rheological behaviour of galactomannan aqueous solutions. Carb. Polym., 59(3), 339-350.
41. Stacy, M. 2000, “Pharmacotherapy for Advanced Parkinson's Disease”, Pharmacotherapy, 20(1), 8-16.
42. Thanvi, B. R. & Lo, T. C. N. 2004, “Long term motor complications of L-dopa: clinical features, mechanisms, and management strategies”, Postgrad. Med. J., 80(946), 452-458.
43. Thirawong, N., Kennedy, R. A. and Sriamornsak, P., 2008. Viscometric study of pectin-mucin interaction and its mucoadhesive bond strength. Carb. Polym., 71(2), 170-179.
44. Vasir, J. K., Tambwekar, K. and Garg, S., 2003. Bioadhesive microspheres as a controlled drug delivery system. International journal of pharmaceutics, 255(1-2), 13-32.
45. Wise, D. L., ed, 1995. Encyclopedic handbook of biomaterials and bioengineering. 1st ed. United Kingdom: Marcel Dekker.
46. Woodley, J., 2001. Bioadhesion: New Possibilities for Drug Administration? Clin. Pharmacokin., 40(2), 77-84.
47. Beun, S.; Glorieux, T.; Devaux, J.; Vreven, J.; Leloup, G. Characterization of nanofilled compared to universal and microfilled composites. Dental Mater. 2007, 23, 51-59.
48. Gojny, F. H.; Wichmann, M. H. G.; Fiedler, B.; Schulte, K. Influence of different carbon nanotubes on the mechanical properties of epoxy matrix composites—a comparative study. Compos. Sci. Technol. 2005, 65, 2300-2313.
49. Gomoll, A. H.; Fitz, W.; Scott, R. D.; Thornhill, T. S.; Bellare, A. Nanoparticulate fillers improve the mechanical strength of bone cement. Acta Orthop. 2008, 79, 421-427.
50. Grant, J.; Blicker, M.; Piquette-Miller, M.; Allen, C. Hybrid films from blends of chitosan and egg phosphatidylcholine for localized delivery of paclitaxel. J. Pharm. Sci. 2005, 94, 1512-1527.
51. Hafner, A.; Lovrić, J.; Voinovich, D.; Filipović-Gr\check{c}ić, J. Melatonin-loaded lecithin/chitosan nanoparticles: Physicochemical characterisation and permeability through caco-2 cell monolayers. Int. J. Pharm. 2009, 381, 205-213.
52. Ho, E. A.; Vassileva, V.; Allen, C.; Piquette-Miller, M. In vitro and in vivo characterization of a novel biocompatible polymer-lipid implant system for the sustained delivery of paclitaxel. J. Control. Release 2005, 104, 181-191.
53. Lim Soo, P.; Cho, J.; Grant, J.; Ho, E.; Piquette-Miller, M.; Allen, C. Drug release mechanism of paclitaxel from a chitosan-lipid implant system: Effect of swelling, degradation and morphology. Eur. J. Pharm. Biopharm. 2008, 69, 149-157.
54. Ndidi C. Ngwuluka, Viness Pillay, Yahya E. Choonara, Girish Modi, Dinesh Naidoo, Lisa C. du Toit, Pradeep Kumar, Valence M. K. Ndesendo and Riaz A. Khan. Fabrication, Modeling and Characterization of Multi-Crosslinked Methacrylate Copolymeric Nanoparticles for Oral Drug Delivery. International Journal of Molecular Sciences. 2011, 12, 6194-6225.
55. Park, J. H.; Jana, S. C. The relationship between nano- and micro-structures and mechanical properties in pmma-epoxy-nanoclay composites. Polymer 2003, 44, 2091-2100.
56. Rapoport, L.; Nepomnyashchy, O.; Verdyan, A.; Popovitz-Biro, R.; Volovik, Y.; Ittah, B.; Tenne, R. Polymer nanocomposites with fullerene-like solid lubricant. Adv. Eng. Mater. 2004, 6, 44-48.
57. Saha, M. C.; Kabir, M. E.; Jeelani, S. Enhancement in thermal and mechanical properties of polyurethane foam infused with nanoparticles. Mater. Sci. Eng. A 2008, 479, 213-222.
58. Sonvico, F.; Cagnani, A.; Rossi, A.; Motta, S.; Di Bari, M. T.; Cavatorta, F.; Alonso, M. J.; Deriu, A.; Colombo, P. Formation of self-organized nanoparticles by lecithin/chitosan ionic interaction. Int. J. Pharm. 2006, 324, 67-73.
59. Zahedi, P.; De Souza, R.; Piquette-Miller, M.; Allen, C. Chitosan-phospholipid blend for sustained and localized delivery of docetaxel to the peritoneal cavity. Int. J. Pharm. 2009, 377, 76-84.
60. Zhang, M. Q.; Rong, M. Z.; Zhang, H. B.; Friedrich, K. Mechanical properties of low nano-silica filled high density polyethylene composites. Polym. Eng. Sci. 2003, 43, 490-500.

1. A pharmaceutical dosage form for the release of at least one pharmaceutically active ingredient, the pharmaceutical dosage form comprising:
a polymer matrix formed from at least two crosslinked polymers;
polymer-lipid nanoparticles formed from at least one polymer and at least one phospholipid and which are incorporated within the polymer matrix; and
at least one pharmaceutically active ingredient.

2. The pharmaceutical dosage form according to claim 1, wherein the polymer-lipid nanoparticles include the pharmaceutically active ingredient.

3. The pharmaceutical dosage form according to claim 1, wherein the polymer matrix includes the pharmaceutically active ingredient.

4. The pharmaceutical dosage form according to claim 1, wherein the two crosslinked polymers are a cationic polymer and an anionic polymer.

5. The pharmaceutical dosage form according to claim 4, wherein the cationic polymer is poly(butyl methacrylate-co-(2-demethylaminoeethyl) methacrylate-co-methyl methacrylate) 1:2:1.

6. The pharmaceutical dosage form according to claim 4, wherein the anionic polymer is sodium carboxymethylcellulose.

7. The pharmaceutical dosage form according to claim 1, wherein the polymer matrix is additionally formed from a third polymer which is a neutral polymer.

8. The pharmaceutical dosage form according to claim 7, wherein the combination of the polymers renders the dosage form gastroretentive.

9. The pharmaceutical dosage form according to claim 7, wherein the neutral polymer is a galactomannon polymer.

10. The pharmaceutical dosage form according to claim 9, wherein the neutral galactomannon polymer is derived from locust bean.

11. The pharmaceutical dosage form according to claim 1, wherein the polymer in the polymer-lipid nanoparticles is methacrylate-co-(2-demethylaminoeethyl) methacrylate-co-methyl methacrylate) 1:2:1.

12. The pharmaceutical dosage form according to claim 1, wherein the polymer in the polymer-lipid nanoparticles is chitosan.

13. The pharmaceutical dosage form according to claim 1, wherein the polymers in the polymer-lipid nanoparticles are methacrylate-co-(2-demethylaminoeethyl) methacrylate-co-methyl methacrylate) 1:2:1 and chitosan.

14. The pharmaceutical dosage form according to claim 1, wherein the phospholipid in the polymer-lipid nanoparticles is lecithin.

15. The pharmaceutical dosage form according to claim 1, wherein the polymer-lipid nanoparticles are formed by cross-linking the polymer and phospholipid with a chelating agent.

16. The pharmaceutical dosage form according to claim 15, wherein the chelating agent is sodium tripolyphosphate.

17. The pharmaceutical dosage form according to claim 1, wherein the polymer matrix swells in a controlled manner when ingested and releases the pharmaceutically active ingredient.

18. The pharmaceutical dosage form according to claim 1, wherein the polymer matrix further includes at least one additive which increases the ability of the matrix to swell.

19. The pharmaceutical dosage form according to claim 18, wherein the additive is a polysaccharide polymer.

20. The pharmaceutical dosage form according to claim 19, wherein the polysaccharide polymer is pullulan.

21. The pharmaceutical dosage form according to claim 1, wherein the polymer matrix further includes at least one excipient.

22. The pharmaceutical dosage form according to claim 1, wherein the pharmaceutically active ingredient is levodopa.

23. The pharmaceutical dosage form according to claim 1, which includes two pharmaceutically active ingredients, wherein the first pharmaceutically active ingredient is incorporated into the polymer-lipid nanoparticles and the second pharmaceutically active ingredient is incorporated into the polymer matrix.

24. The pharmaceutical dosage form according to claim 1, for use in the treatment of Parkinson's disease.

25. A method of preparing a pharmaceutical dosage form for the release of a pharmaceutically active ingredient, the method comprising the steps of:
synthesizing a polymer matrix by crosslinking at least two polymers;
synthesizing polymer-lipid nanoparticles from at least one polymer and at least one phospholipid; and
incorporating the polymer-lipid nanoparticles into the polymer matrix;
wherein the pharmaceutically active ingredient is added to either the polymer matrix and/or to the polymer-lipid nanoparticles.

26. The method according to claim 25, wherein the pharmaceutically active ingredient is added to the polymer-lipid nanoparticles.

27. The method according to claim 25, wherein the pharmaceutically active ingredient is added to the polymer matrix.

28. The method according to claim 25, wherein the two crosslinked polymers are a cationic polymer and an anionic polymer.

29. The method according to claim 28, wherein the cationic polymer is methacrylate-co-(2-demethylaminoeethyl) methacrylate-co-methyl methacrylate) 1:2:1 and wherein the anionic polymer is sodium carboxymethylcellulose.

30. (canceled)

31. The method according to claim 29, wherein the ratio of (methacrylate-co-(2-demethylaminoeethyl) methacrylate-co-methyl methacrylate) 1:2:1 to Sodium Carboxymethylcellulose is 0.5:1.

32.-50. (canceled)

51. The pharmaceutical dosage form according to claim 5, wherein the anionic polymer is sodium carboxymethylcellulose.

52. The pharmaceutical dosage form according to claim 8, wherein the neutral polymer is a galactomannon polymer.

53. The pharmaceutical dosage form according to claim 52, wherein the neutral galactomannon polymer is derived from locust bean.
